(19) United States
(12) Patent Application Publication
(10) Pub. No.: US 2023/0280275 A1
(43) Pub. Date: Sep. 7, 2023

(54) PHOTOACTIVATABLE VIBRATIONAL PROBES AND USES THEREOF

(71) Applicant: California Institute of Technology, Pasadena, CA (US)

(72) Inventors: Lu Wei, Pasadena, CA (US); Jiajun Du, Pasadena, CA (US)

(21) Appl. No.: 17/960,629

(22) Filed: Oct. 5, 2022

Related U.S. Application Data
(60) Provisional application No. 63/252,930, filed on Oct. 6, 2021.

(51) Int. Cl. G01N 21/65

(52) U.S. Cl. CPC G01N 21/65; G01N 2021/656; G01N 2500/10

(57) ABSTRACT

Disclosed herein include a photoactivatable vibrational probe, when photoactivated, capable of forming a Raman probe for detection by Raman scattering. In some embodiments, the photoactivatable vibrational probe has a structure of Formula I. Disclosed herein also includes methods of using the photoactivatable vibrational probe for live-cell multiplexed imaging and tracking.

### RELATED APPLICATIONS

The present application claims priority under 35 U.S.C. §119(e) to U.S. Provisional Application No. 63/252,930, filed on Oct. 6, 2021, the content of which is herein expressly incorporated by reference in its entirety.

### BACKGROUND

### Field

The present disclosure relates generally to the field of imaging technology, and in particular Raman scattering imaging.

### Description of the Related Art

Photoactivatable probes, with high-precision spatial and temporal control, have largely advanced bioimaging applications, particularly for fluorescence microscopy. However, fluorescent probes such as organic dyes, fluorescent proteins or quantum dots are all relatively bulkier in size than small biomolecules, thus often severely compromising the native biochemical or biophysical properties of these fluorophore-labeled small biomolecules inside live cells.

Compared to fluorescence, Raman microscopy has several unique advantages due to its nature of detecting vibrational motions of chemical bonds instead of probing the electronic state transitions of conjugated fluorophores. Recent coupling of the nonlinear stimulated Raman scattering imaging with Raman probes has helped address several fundamental challenges in fluorescence microscopy, such as for imaging small biomolecules and for super-multiplex imaging. Nevertheless, light-activatable Raman probes, which would enable photoactivatable Raman imaging while preserving multiplexity and small-size features, remain less explored. Thus, there remains a need for photoactivatable Raman probes that can offer superior sensitivity and specificity as well as multiplexity for tracking complex cellular dynamics and interactions.

### SUMMARY

A photoactivatable vibrational probe, having a structure according to Formula I:

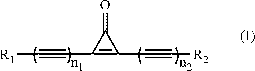

wherein R₁ and R₂ independently represent a monocyclic or polycyclic, aromatic or heteroaromatic ring; n₁ and n₂ are independently 0 or 1, and wherein the photoactivatable vibrational probe upon photoactivation forms a vibrational probe comprising a planar alkyne generated from the cyclopropenone of Formula I.

In some embodiments, the photoactivatable vibrational probe has a structure according to Formula II:

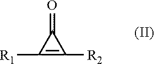

In some embodiments, R₁ and R₂ each is a phenyl group. In some embodiments, one or more carbon atoms of one or both phenyl groups are substituted with a substituent selected from the group consisting of: alkyl, aryl, heteroaryl, halogen, hydroxyl, alkyl alcohol, carboxyl, alkoxyl, aryloxyl, thio, alkythio, amino, alkylamino, aldehyde, alkenyl, alkynyl, benzyl, carboxamide, azo, ester, carbonyl, nitrile, nitro, phenyl, sulfonyl, sulfinyl, and a combination thereof.

In some embodiments, the photoactivatable vibrational probe has a formula of:

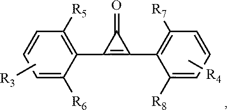

wherein R₃ and R₄ are independently a hydrogen, a hydroxyl group, an alkyl group, an alkoxy group, an amide, a ketone, a carboxyl group, a halogen, an ether, or an ester; R₅, R₆, R₇ and R₈ are independently hydrogen, alkyl, aryl, heteroaryl, halogen, hydroxyl, alkyl alcohol, carboxyl, alkoxyl, aryloxyl, thio, alkythio, amino, and alkylamino, aldehyde, alkenyl, alkynyl, benzyl, carboxamide, azo, ester, carbonyl, nitrile, nitro, phenyl, sulfonyl, or sulfinyl.

In some embodiments, the photoactivatable vibrational has a formula of:

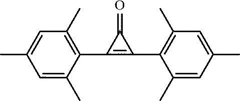

In some embodiments, one of the methyl groups in one or both of the phenyl group is substituted with a hydroxyl group or a hydrogen.

The photoactivatable vibrational probe, in some embodiments, can further comprises a targeting moiety. The targeting moiety can be covalently attached to the photoactivatable vibrational probe via a hydroxyl group.

In some embodiments, the photoactivatable vibrational probe has a formula of:

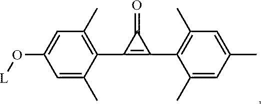

wherein L represents a targeting moiety.

In some embodiments, the photoactivatable vibrational probe has one of the following structures:

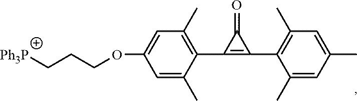

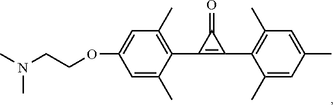

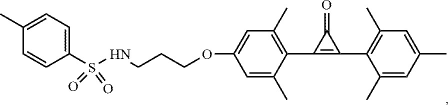

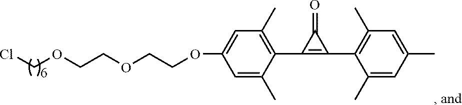

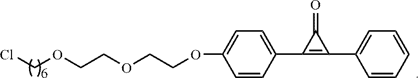

In some embodiments, the photoactivatable vibrational probe comprises at least one ¹³C atom. In some embodiments, the photoactivatable vibrational probe has a formula of

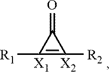

wherein one or both of X₁ and X₂ is a ¹³C atom.

Also disclosed herein includes a method of imaging a biological material. The method, in some embodiments, comprises: introducing at least one of the photoactivatable vibrational probes disclosed herein to the biological material; activating the at least one photoactivatable vibrational probe with light to generate at least one vibrational probe; and detecting the at least one vibrational probe using Raman scattering.

In some embodiments, the contacting comprises introducing two or more photoactivatable vibrational probes into the biological material, wherein optionally the two or more photoactivatable vibrational probes when activated exhibit different Raman peaks. In some embodiments, the at least one photoactivatable vibrational probe is attached to a targeting moiety. In some embodiments, the target moiety is capable of specifically binding to a cell marker, an organelle, proteins, sugars, lipids, nucleic acids, metabolites, or any combination thereof. In some embodiments, the targeting moiety binds to a receptor of an organelle or a cell in the biological material. In some embodiments, the targeting moiety targets mitochondria, lysosome, endoplasmic reticulum, lipid droplet, or any combination thereof.

In some embodiments, the two or more photoactivatable vibrational probes each is attached to a targeting moiety that targets a different cell type, a different organelle or a different biomolecule in the biological material. In some embodiments, activating the at least one photoactivable vibrational probe comprises applying light having a wavelength from about 200 nm to about 1500 nm through one-photon or multi-photon absorption to the biological material. In some embodiments, the at least one vibrational probe each comprises an alkyne after activation. The alkyne can be, for example, isotopically modified. In some embodiments, the at least one vibrational probe is imaged using a stimulated Raman scattering (SRS) imaging procedure or spontaneous Raman imaging procedure. In some embodiments, detecting the at least one vibrational probe using Raman scattering comprises detecting the at least one vibrational probe at a first time point and detecting the at least one vibrational probe at a second time point as a pulse-chase experiment, wherein the first time point is different from the second time point.

The method, in some embodiments, comprises comparing a first image obtained from detecting the at least one vibrational probe at the first time point and a second image obtained from detecting the at least one vibrational probe at the second time point. In some embodiments, the biological material comprises live cells, biomolecules, a cell line, cells constituent derived from or located in a mammal, organs, living organism, biological tissues, a biological fluid, or a combination thereof. In some embodiments, the biological material comprises a tissue section for use in Raman imaging application. The tissue can be, for example, a connective tissue, a blood and lymph tissue, a nervous tissue (e.g., a central nervous system (CNS) and/or a peripheral nervous system (PNS) tissue), an epithelial tissue (e.g., skin tissue), or a muscle tissue. In some embodiments, the biological material comprises healthy, diseased or malignant tissue. The contacting can occur, for example, in vivo, ex vivo, in vitro, or a combination thereof.

Disclosed therein includes a composition for Raman imaging, comprising at least one photoactivatable vibrational probe disclosed herein. Also disclosed herein includes a method of imaging a subject. The method, in some embodiments, comprises, administering one or more of the compositions disclosed herein for Raman imaging to the subject; activating the at least one photoactivatable vibrational probe with light to generate at least one vibrational probe; and imaging the subject using Raman scattering. In some embodiments, two or more photoactivatable vibrational probes are administered to the subject, the two or more photoactivatable probes when activated exhibiting different Raman peaks. In some embodiments, the at least one photoactivatable vibrational probe is attached to a targeting moiety that directs the at least one photoactivatable vibrational probe to a desired imaging site. In some embodiments, activating the at least one photoactivatable vibrational probe comprises applying absorbing light. Imaging the subject can comprise imaging the subject using Raman scattering at a first time point about or prior to the initiation of a treatment.

In some embodiments, the method comprises applying the treatment to the subject and imaging the subject using Raman scattering at a second time point after the initiation of the treatment. In some embodiments, the subject is imaged using a SRS imaging procedure. In some embodiments, imaging the subject using Raman scattering comprises detecting the at least one vibrational probe using SRS. The administration can be, for example, a systematic administration. In some embodiments, the administration is an oral delivery or an intravenous injection. The subject can be a mammal, for example a human.

Disclosed herein includes a method for controlled releasing of carbon monoxide (CO) into a biological material. The method, in some embodiments, comprises: introducing one or more of the photoactivatable vibrational probe disclosed herein to the biological material; and activating the photoactivatable vibrational probe with light, thereby releasing CO into the biological material. In some embodiments, the method comprises detecting the released CO.

### BRIEF DESCRIPTION OF THE DRAWINGS

FIGS. 1A-D provide characterizations of exemplary non-limiting model systems for photoactivatable generation of alkynes. FIG. 1A depicts exemplary structures and reactions for four precursors of alkynes, including photo-DIBO, 1-cyclo (1), 2-cyclo (3) and 3-cyclo (5) that can potentially undergo UV-activated alkyne generation. FIG. 1B are graphs showing spontaneous Raman and hyperspectral SRS spectra for 10 mM precursors and products shown in FIG. 1A. FIG. 1C is a graph showing UV-Vis absorption spectra of all four precursors shown in FIG. 1A (20 uM, in DMSO). Vertical lines indicate the positions of 365 nm and 405 nm which were used for photoactivation. FIG. 1D are graphs showing times-series spontaneous Raman spectra for monitoring the photoactivation kinetics for 1-cyclo (1, top) and 2-cyclo (3, bottom) under 365 nm illumination (15 mW/cm²). Inserts show the increase kinetics of alkyne peaks (2226 cm⁻¹).

FIGS. 2A-D provide results from molecular engineering of an exemplary non-limiting photoactivatable probe described herein: 1-cyclo (1). FIG. 2A is a graph showing HPLC trace of 1-cyclo (1) after incubating in 10 mM cysteine (PBS) for designated time at 37° C. FIG. 2B depicts an exemplary design principle of incorporating methyl groups to shield cyclopropenones from nucleophilic attack. FIG. 2C is a graph showing HPLC trace of 8 after incubating in 10 mM cysteine (PBS) for designated time at 37° C. FIG. 2D is a graph showing spontaneous Raman comparisons for 40 mM solutions of 9 (red dashed), 10 (red solid, generated by illuminating 9 with 365 nm), 11 (blue solid) and 1-yne (2, black solid).

FIG. 3 depicts exemplary structures and images of methylated cyclopropenone probes for photoactivated SRS imaging of organelle targets including mitochondria (Me-Mito, 12, a), lysosomes (Me-Lyso, 13, b), endoplasmic reticulum (Me-ER, 14, c) and lipid droplets (Me-LD, 15, d) in live HeLa cells. Fluorescence imaging with co-stained commercial markers were used to validate the labeling specificity of SRS probes via co-localization. Scale bar: 10 µm.

FIG. 4 shows an exemplary three-color photoactivatable organelle imaging with Me-Mito-¹³C (16), Me-Lyso (13) and Cyclo-LD (17) on live HeLa cells. Panel (a): Chemical structures of Me-Mito-¹³C (16), Me-Lyso (13) and Cyclo-LD (17). Panel (b): The normalized spontaneous Raman spectra of Me-Mito-¹³C (16), Me-Lyso (13) and Cyclo-LD (17) after UV-uncaging. Panel (c): SRS images of live HeLa cells before and after 405 nm illumination, labeled with Me-Mito-¹³C (16, 2125 cm⁻¹), Me-Lyso (13, 2205 cm⁻¹) or Cyclo-LD (17, 2226 cm⁻¹), respectively. 2940 cm⁻¹ images for CH₃ outline the cell morphology. Panel (d): Three-color photoactivatable Raman imaging of the same set of live HeLa cells co-labeled with Me-Mito-¹³C (16), Me-Lyso (13) and Cyclo-LD (17). Scale bar: 10 µm.

FIG. 5 shows exemplary results from subcellular and single-cell multiplex tracking with pulse-chase photoactivatable imaging. Panel (a): Pulse-chase photoactivation and tracking for a selected subcellular region of a single cell (indicated by white dashed box) from Me-Mito-¹³C (16) labeled live HeLa cells. Pulse (Mito₀ ₘᵢₙ) shows SRS imaging at 2125 cm⁻¹ immediately after 405 nm photoactivation, and chase (Mito₁₀ ₘᵢₙ) presents the distribution from the same field-of-view imaged after 10 minutes. The overlay of pulse (magenta) and chase (green) is shown in Merge (Mito₀ ₘᵢₙ_{/10} ₘᵢₙ). Panel (b): Pulse-chase SRS images for the selectively activated single cell (CH₃, indicated by white dashed box) from live HeLa cells co-labeled with Me-LD (15) and Me-Mito-¹³C (16). Two-color SRS images (2125 cm⁻¹ for mitochondria, Mito; 2205 cm⁻¹ for lipid droplets, LD) present the pulse (0 h) and chase (1 h, 2 h) distributions with the magnified images showing the selected cell together with its neighboring cell. The numbers in the magnified mitochondria images indicate the relative signal percentages of the top and the bottom cells. Scale bar: 10 µm.

FIG. 6 are graphs showing UV-Vis absorption spectra of 20 µM photo-DIBO, 1-cyclo (1), 2-cyclo (3) and 3- cyclo (5) and their corresponding alkyne products, shown in FIG. 1A, in DMSO.

FIG. 7 provides exemplary results from SRS imaging of live HeLa cells incubated with 1-cyclo (1, 20 µM, 30 minutes of labeling, top) and Cyclo-Mito (7, 20 µM, 30 minutes of labeling, bottom) at the CH₃ (2940 cm⁻¹) channel, and the expected uncaged alkyne channel (2226 cm⁻¹) before and after 405 nm illumination. Scale bar: 10 µm.

FIG. 8 is a graph showing HPLC trace of Cyclo-Mito (7) after incubating in 10 mM cysteine (PBS) for designated duration at 37° C.

FIG. 9 is a graph showing UV-Vis absorption spectra of 20 uM 1-cyclo (1, black) and 9 (gray) in DMSO.

FIG. 10 is a graph showing HPLC trace of Me-Mito (12) after incubating in 10 mM cysteine (PBS) for designated duration at 37° C.

FIG. 11 shows exemplary magnified images of FIG. 3, panel a (set 1) and two independent sets of correlative SRS imaging of Me-Mito (12) and fluorescence imaging of Mitotracker deep red in live HeLa cells. The correlation coefficients (Pearson’s R value) for set 1, set 2 and set 3 (Fl/2225 cm⁻¹ channel) are 0.85, 0.82 and 0.88 respectively. Scale bar: 10 µm.

FIG. 12 provides exemplary characterizations of Me-Mito (12) photoactivation by SRS imaging lasers. 10 consecutive frames of SRS images (2205 cm⁻¹, The representative 1st, 5th, 10th images are shown) taken on live HeLa cells (CH₃ SRS imaging at 2940 cm⁻¹ outlines the cell morphology) after incubation with 20 µM Me-Mito (12) for 30 minutes. SRS imaging at 2205 cm⁻¹ after full photoactivation (405 nm activation) is also shown for 100% intensity benchmark for Me-Mito (12) labeling. The relative SRS activated intensity over SRS laser illumination time is plotted in the bottom (the SRS intensity after 405 nm activation is set as 100%). Average frame activation is 1.4% at the chosen condition: OPO and Stokes powers are 25 mW and 60 mW, respectively; and pixel dwell time is 80 µs/pixel for each image frame. Scale bar: 10 µm.

FIG. 13 is a table showing intensity gain per frame under different power combinations of the OPO and Stokes lasers with a pixel dwell time of 80 µs/pixel. Gray-colored conditions indicate the selection of our SRS imaging conditions with an average per frame activation less than 1.5%.

FIG. 14, panel a is a plot for intensity gain per frame at different power conditions shown in FIG. 13. The size of the cycles is proportional to the intensity gain per frame. FIG. 14, panel b is a graph showing SRS Intensity gain per imaging frame (Intensity gain/frame) from time-series SRS imaging is plotted on live HeLa cells incubated with the indicated concentration of Me-Mito (12) probe for 30 minutes. The intracellular labeling concentration is calculated by the signal level after final 405 nm activation and benchmarked with the SRS standard curve from the uncaged probe solutions. SRS laser powers: 40 mW for OPO and 40 mW for Stokes, respectively. n = 4 independent experiments. Data shown as mean ± SEM.

FIG. 15 shows exemplary magnified images of FIG. 3, panel b (set 1) and two independent sets of correlative SRS imaging of Me-Lyso (13) and fluorescence imaging of Lysoview 488 in live HeLa cells. The correlation coefficients (Pearson’s R value) for set 1, set 2 and set 3 (Fl/2205 cm⁻¹ channel) are 0.60, 0.67 and 0.62 respectively. We note that, the lowered Pearson’s R are mainly due to the active movements of lysosomes in the live cells, from the 3-5 min gap (2 minutes of 405 nm laser illumination + SRS laser tuning time) between acquiring fluorescence and photoactivated SRS images. Scale bar: 10 µm.

FIG. 16 shows exemplary magnified images of FIG. 3, panel c (set 1) and two independent sets of correlative SRS imaging of Me-ER (14) and fluorescence imaging of ER tracker red in live HeLa cells. The correlation coefficients (Pearson’s R value) for set 1, set 2 and set 3 (Fl/2205 cm⁻¹ channel) are 0.86, 0.80 and 0.90 respectively. Scale bar: 10 µm.

FIG. 17 shows exemplary magnified images of FIG. 3, panel d (set 1) and two independent sets of correlative SRS imaging of Me-LD (15) and fluorescence imaging of Lipid spot 610 in live HeLa cells. The correlation coefficients (Pearson’s R value) for set 1, set 2 and set 3 (Fl/2205 cm⁻¹ channel) are 0.54, 0.54 and 0.50 respectively. It is noted that, the lowered Pearson’s R are mainly due to the active movements of lipid droplets in the live cells, from the 3-5 min gap (2 minutes of 405 nm laser illumination + SRS laser tuning time) between acquiring fluorescence and photoactivated SRS images. The colocalization is much improved between the 2205 cm⁻¹ channel and the label-free CH₂ lipid channel (2845 cm⁻¹), which are acquired within 1-min time interval. Scale bar: 10 µm.

FIG. 18 provides an exemplary characterization of an exemplary photoactivatable probe cyclo-LD (17) and its suitability for live-cell lipid droplet imaging. Panels (a-b) are plots showing the stability of Cyclo-LD (17) in 10 mM cysteine in PBS (a), and DMEM (b) at 37° C. Panel (c) includes two independent sets of correlative SRS imaging of Cyclo-LD (17) and fluorescence imaging of Lipid spot 610 in live HeLa cells. The correlation coefficients (Pearson’s R value) for set 1 and set 2 (Fl/2226 cm⁻¹ channel) are 0.50 and 0.76 respectively, which is again lowered due to active droplet movements. Scale bar: 10 µm.

FIG. 19 demonstrates minimum chemical toxicity of cyclopropenone dyes. Control fluorescence images for live/dead cell-viability assay for live HeLa cells (calcein-AM, green, as live cell indicator) and fixed cells (EthD-1, red, as dead cell indicator). The live HeLa cells were labeled with the indicated probe for the indicated time before performing live/dead cell-viability assay. Scale bar: 10 µm.

FIG. 20 illustrates labeling signal and specificity evaluation during 2 hours of time lapse imaging in photoactivated Me-Mito-¹³C (16) labeled live HeLa cells. Panel (a) is a plot showing quantification of retained signals after 1 hour and 2 hours imaging. The values of signals at 0 h were used as 100% signal reference. n = 6 independent experiments. Data shown as mean ± SEM. Panel (b) includes a series of representative time lapse imaging. The magnified images of the bottom cell (indicated by the dashed white box) are shown. Scale bar: 10 µm.

FIG. 21 provides two independent sets of cell images (panels a and b) from multiplex imaging analysis for interactions between lipid droplets and lysosomes during mTOR inhibition on selectively photoactivated live HeLa cells. The cells were activated by 405 nm (selective activation of two cells indicated by the dashed white boxes in (panel a), zoom-in activation in (panel b)) immediately after rapamycin treatment (0 min).

FIG. 22 provides HRMS, ¹H NMR (400 MHz, CD₂Cl₂) and ¹³C NMR (400 MHz, CD₂Cl₂) spectra of 2-cyclo (3).

FIG. 23 provides HRMS and ¹H NMR (400 MHz, CD₂Cl₂) spectra of Cyclo-Mito (7).

FIG. 24 provides HRMS, ¹H NMR (400 MHz, CD₂Cl₂) and ¹³C NMR (400 MHz, CD₂Cl₂) spectra of Cyclo-LD (17).

FIG. 25 provides HRMS, ¹H NMR (400 MHz, CD₂Cl₂ with CD₃OD as co-solvent) and ¹³C NMR (400 MHz, CD₂Cl₂ with CD₃OD as co-solvent) spectra of 8.

FIG. 26 provides HRMS and ¹H NMR (400 MHz, CD₂Cl₂ with CD₃OD as co-solvent) spectra of the isomer of 8.

FIG. 27 provides HRMS, ¹H NMR (400 MHz, CD₂Cl₂) and ¹³C NMR (400 MHz, CD₂Cl₂) spectra of Me-Mito (12).

FIG. 28 provides HRMS, ¹H NMR (400 MHz, CD₂Cl₂) and ¹³C NMR (400 MHz, CD₂Cl₂) spectra of Me-Lyso (13).

FIG. 29 provides HRMS, ¹H NMR (400 MHz, CD₂Cl₂) and ¹³C NMR (400 MHz, CD₂Cl₂) spectra of Me-ER (14).

FIG. 30 provides HRMS, ¹H NMR (400 MHz, CD₂Cl₂) and ¹³C NMR (400 MHz, CD₂Cl₂) spectra of Me-LD (15).

FIG. 31 provides ¹H NMR (400 MHz, CD₂Cl₂) and ¹³C NMR (400 MHz, CD₂Cl₂) spectra of I1.

FIG. 32 provides ¹H NMR (400 MHz, CD₂Cl₂) and ¹³C NMR (400 MHz, CD₂Cl₂) spectra of I2.

FIG. 33 provides ¹H NMR (400 MHz, CD₂Cl₂) and ¹³C NMR (400 MHz, CD₂Cl₂) spectra of I4.

FIG. 34 provides HRMS, ¹H NMR (400 MHz, CD₂Cl₂) and ¹³C NMR (400 MHz, CD₂Cl₂) spectra of Me-Mito-¹³C (16).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Photoactivation, which allows for precise spatial-temporal control of target molecules, has found wide applications in biological research, such as for the delivery of therapeutic agents and the modulation of cellular chemistry and physiology. For microscopy, the invention of photoactivable fluorescent probes, which can be converted from the non-fluorescent to the fluorescent state by light with photolabile groups, has facilitated the development of ground-breaking localization-based super-resolution microscopy, such as photoactivated localization microscopy (PALM). In addition, these fluorescent probes have opened new ways to study the dynamics of cell migration and the interactions of biomolecules at the targeted subcellular locations and time periods.

Complementary to fluorescence, Raman microscopy also offers superior optical imaging capabilities. Notably, the recent coupling of the nonlinear stimulated Raman scattering (SRS) imaging with Raman probes, has helped address several fundamental challenges in fluorescence microscopy, such as for imaging small biomolecules and for super-multiplex imaging in live cells. Compared to fluorescence, Raman microscopy has several unique advantages due to its nature of detecting vibrational motions of chemical bonds instead of probing the electronic state transitions of conjugated fluorophores. First, the sizes of Raman probes are usually much smaller than those of fluorophores. This introduces much less physical perturbation when tagging biomolecules. Second, the Raman peak linewidth is about 50-100 times narrower compared to the broad absorption and the emission peaks from fluorophores. Third, Raman signals don’t involve electronic excitation, and are resistant to photobleaching or environmental quenching, and hence are better suited for quantifications. These features allow for super-multiplexed optical imaging with high information throughput for interrogating the complex biology. The designed matching polyynes and electronic pre-resonance Raman dye palettes (e.g. the CARBOW and the MARS dye palettes) have achieved more than 20-plex SRS imaging with reported sensitivity close to what is offered by confocal fluorescence.

Electronic pre-resonance SRS dye based enzyme-activatable and photoswitchable Raman probes have been recently reported, demonstrating the remarkable multiplexable or photo-controllable features for functional cellular imaging, respectively. However, enzyme-activatable probes lack the photo-controllable features. While reversibly switchable Raman probes share certain imaging functions with photoactivatable probes, their additional off-switching pathway was shown to be triggerable by the SRS imaging laser. Additionally, their off-channel signals may also limit the resolvable Raman colors.

Light-activatable Raman probes would enable photoactivatable Raman imaging while preserving multiplexity and small-size features. This new functional Raman imaging would be highly beneficial to multi-component imaging and tracking. However, this category of Raman probes remains unexplored.

Provided herein includes engineered photoactivatable Raman probes/vibrational probes based on cyclopropenone caging for live-cell imaging and tracking. The terms “Raman probe” and “vibrational probe” can be used interchangeable herein. The photo-releasable alkyne scaffold is adopted for engineering the photoactivatable Raman probes described herein since alkynes are small and highly Raman-sensitive in the cell silent spectral region (1800-2800 cm⁻¹). In addition, alkynes can readily offer multiplexable Raman colors through proper isotope editing or chemical-group capping. The photoreaction that generates alkyne bonds can serve as a platform for constructing photoactivatable Raman imaging probes. The photoactivatable Raman probes disclosed herein exhibit enhanced chemical stability in live-cell environment and improved Raman sensitivity. Described herein also include methods of using the photoactivatable Raman probes for imaging live biological materials (e.g., cells, organelles, biomolecules, organisms, tissues, organs, and/or subject) as well as multiplexed photoactivated imaging and tracking at both subcellular and cellular levels to monitor the dynamic migration and interactions of the cellular contents.

Disclosed herein includes a photoactivatable vibrational probe. In some embodiments, the photoactivatable vibrational probe has a structure according to Formula I:

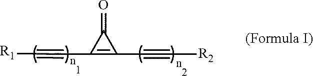

wherein R₁ and R₂ independently represent a monocyclic or polycyclic, aromatic or heteroaromatic ring; n₁ and n₂ each independently is 0 or 1. The photoactivatable vibrational probe upon photoactivation forms a vibrational probe comprising a planar alkyne generated from the cyclopropenone of Formula I.

Disclosed herein also include a method of imaging a biological material. In some embodiments, the method comprises introducing at least one photoactivatable vibrational probe described herein into the biological material. The method can also comprise activating the at least one photoactivatable vibrational probe with light to generate at least one vibrational probe and detecting the at least one vibrational probe using Raman scattering. Raman scattering can be spontaneous Raman imaging or a stimulated Raman scattering imaging. In some embodiments, the biological materials comprise live cells, organelles, living organism, tissues, organs, and/or biomolecules such as lipids, proteins, nucleic acids, sugars or metabolites.

Disclosed herein also includes a method of imaging a subject. The method can comprise administering a composition comprising at least one photoactivatable vibrational probe described herein to the subject. The method can also comprise activating the at least one photoactivatable vibrational probe with light to generate at least one vibrational probe and imaging the subject using Raman scattering. In some embodiments, the subject is a human.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

As used herein, the term “about” means plus or minus 5% of the provided value.

The term “alkyl group” means a saturated linear or branched monovalent hydrocarbon chain having a specified number of carbon atoms including, for example, methyl, ethyl, n-propyl, isopropyl, tert-butyl, amyl, heptyl, and the like. The term “alkyl” can also refer to the radical of saturated aliphatic groups (i.e., an alkane with one hydrogen atom removed), including straight-chain alkyl groups, branched-chain alkyl groups, cycloalkyl (alicyclic) groups, alkyl-substituted cycloalkyl groups, and cycloalkyl-substituted alkyl groups. The term “alkyl” as used herein can include both “unsubstituted alkyls” and “substituted alkyls,” the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocycle, aralkyl, or an aromatic or heteroaromatic moiety. In some embodiments, an alkyl used herein can refer to an alkyl group having from one to ten carbons in its backbone structure, preferably one to six carbons (C₁₋₆ alkyl).

The term “alkenyl group” refers to an unsaturated, linear or branched monovalent hydrocarbon group with one or more olefinically unsaturated groups (i.e., carbon-carbon double bonds), such as a vinyl group.

The term “alkynyl group” or “alkyne” refers to an unsaturated, linear or branched monovalent hydrocarbon group with one or more carbon-carbon triple bonds (—C═C—). In some embodiments, an “alkynyl” group contains from 2 to 24 carbon atoms. Representative straight chain and branched alkynyl groups include acetylenyl, propynyl, 1-butynyl, 2-butynyl, 1-pentynyl, 2-pentynyl, and 3-methyl-1-butynyl. In some embodiments, an alkynyl group is a planar alkynyl group, rather than a cyclic alkynyl group.

The term “cyclic group” refers to any chemical group in which one or more series of atoms is connected to forma a ring. A cyclic group can be an alicyclic group, aromatic group, or heterocyclic group. A cyclic group can be monocyclic, bicyclic or polycyclic. A cyclic group can also be a heterocyclic group comprising one or more heteroatoms in the ring group.

The term “alicyclic group” means a cyclic hydrocarbon group having properties resembling those of aliphatic groups.

The term “aryl” as used herein can refer to C₅-C₁₀-membered aromatic, heterocyclic, fused aromatic, fused heterocyclic, biaromatic, or bihetereocyclic ring systems. “Aryl” can includes 5- to 7-membered single-ring aromatic groups, preferably a 6-membered ring. In some embodiments, each atom of the ring is carbon. In some embodiments, the aromatic group can include one or more heteroatoms (e.g., nitrogen, oxygen, sulfur, etc). Those aryl groups having heteroatoms in the ring structure can also be referred to as “heteroaromatic group”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, and combinations thereof.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfuydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “acyl” is art-recognized and refers to a group represented by the general formula hydrocarbylC(O)-, preferably alkylC(O)-.

The term “acylamino” is art-recognized and refers to an amino group substituted with an acyl group and may be represented, for example, by the formula hydrocarbylC(O)NH-.

The term “acyloxy” is art-recognized and refers to a group represented by the general formula hydrocarbylC(O)O-, preferably alkylC(O)O-.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “hydroxyalkyl” refers to an alkyl group having a hydroxyl attached thereto. Representative hydroxyalkyl groups include hydroxyethyl (-CH₂CH₂OH), hydroxypropyl, hydroxybutyl, hydroxypentyl, and the like.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl—O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “amine” and “amino” are art-recognized and refer to both unsubstituted and substituted amines and salts thereof, e.g., a moiety that can be represented by

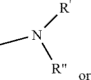

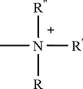

wherein R, R′, and R′′ each independently represent a hydrogen, alkyl, substituted alkyl, aryl, substituted aryl, aryl alkyl, substituted aryl alkyl, heteroaryl, and substituted heteroaryl. A substituted amine being an amine group wherein R′ or R′′ is other than hydrogen. In a primary amino group, both R′ and R′′ are hydrogen, whereas in a secondary amino group, either, but not both, R′ or R′′ is hydrogen. In addition, the terms “amine” and “amino” can include protonated and quatemized versions of nitrogen.

The term “amino alkyl”, as used herein, refers to an alkyl group substituted with an amino group.

The term “aralkyl”, as used herein, refers to an alkyl group substituted with an aryl group.

The term “carboxyalkyl” as used herein refers to a group having the general formula —(CH₂)ₙCOOH, where n is 1-18.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, and the like.

The terms “heterocyclyl”, “heterocycle”, and “heterocyclic” refer to substituted or unsubstituted non-aromatic ring structures, preferably 3- to 10-membered rings, more preferably 3-to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have an =O or =S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a =O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

As used herein, the term “Raman scattering” refers to a spectroscopic technique used to observe vibrational, rotational, and other low-frequency modes in a system. It relies on inelastic scattering, or Raman scattering, of monochromatic light, usually from a laser in the visible, near infrared, or near ultraviolet range. The laser Light interacts with molecular vibrations, phonons or other excitations in the system, resulting in the energy of the laser photons being shifted up or down.. The shift in energy gives information about the vibrational modes in the system. A variety of optical processes, both linear and nonlinear in light intensity dependence, are fundamentally related to Raman scattering. As used herein, the term “Raman scattering” includes, but is not limited to, “stimulated Raman scattering” (SRS), “spontaneous Raman scattering”, “coherent anti-Stakes Raman scattering” (CARS), “surface-enhanced Raman scattering” (SERS), “Tip-enhanced Raman scattering” (TERS) or “vibrational photoacoustic tomography”.

As used herein, “treatment” refers to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Subjects in need of treatment include those already affected by a disease or disorder or undesired physiological condition as well as those in which the disease or disorder or undesired physiological condition is to be prevented.

As used herein, a “subject” refers to an animal for whom a diagnosis, treatment, or therapy is desired. I some embodiments, the subject is a mammal. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a primate. In some embodiments, the mammal is a human. In some embodiments, the mammal is not a human.

### Photoactivable Raman Probes

Provided herein include photoactivatable Raman probes designed based on cyclopropenone caging for live-cell multiplexed imaging and tracking using Raman scattering. The photoactivatable Raman probes or caged Raman probes can form a planar alkyne bond from the cyclopropenone of the photoactivatable Raman probe when illuminated with an appropriate wavelength (i.e. after uncaging). In some embodiments, the fast photochemically generated alkynes from cyclopropenones enable background-free Raman imaging with desired photo-controllable features through, for example, isotope editing or chemical-group capping. The photoactivatable Raman probes described herein can not only offer combined features of quick activation, small sizes and desirable Raman selectivity and sensitivity, but also demonstrate substantially chemical stability in the cellular environment and enhanced Raman signals.

A photoactivatable Raman probe can have a structure according to Formula I:

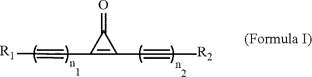

wherein R₁ and R₂ independently represent a monocyclic or polycyclic, aromatic or heteroaromatic ring; n₁ and n₂ each independently is 0 or 1. The photoactivatable vibrational probe upon photoactivation forms a vibrational probe comprising a planar alkyne generated from the cyclopropenone of Formula I. The photoactivatable vibrational probe upon photoactivation can be converted into a Raman imaging probe (also referred to as a vibrational probe) that can be detected by Raman scattering. In some embodiments, the converted vibrational probe comprises one or more chemical bonds that vibrate in the cell-silent Raman window (1800 - 2800 cm⁻¹) in which no endogenous molecules vibrate. The vibrational probe can exhibit at least one Raman peak in the region of 1800 - 2800 cm⁻¹. In some embodiments, upon photoactivation (e.g., with ultra-violet light) the photoactivatable vibrational probe described herein forms a vibrational probe comprising a planar alkyne generated from the cyclopropenone of Formula I for detection by Raman scattering.

In some embodiments, a photoactivatable Raman probe has a structure according to Formula II:

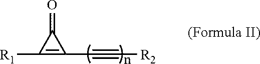

wherein R₁ and R₂ independently represent a monocyclic or polycyclic, aromatic or heteroaromatic ring; and n is 0 or 1.

In some embodiments, one or both of R₁ and R₂ in Formula I and II is a phenyl group. In some embodiments, one or more carbon atoms of one or both phenyl groups can be substituted. For example, one or more carbon atoms of one or both phenyl groups can be substituted with a substituent selected from the group consisting of: alkyl, aryl, heteroaryl, halogen, hydroxyl, alkyl alcohol, carboxyl, alkoxyl, aryloxyl, thio, alkythio, amino, alkylamino, aldehyde, alkenyl, alkynyl, benzyl, carboxamide, azo, ester, carbonyl, nitrile, nitro, phenyl, sulfonyl, sulfinyl, and a combination thereof.

In some embodiments, upon photoactivation, the photoactivatable vibrational probe can be converted to a vibrational probe or Raman probe comprising at least one planar alkyne that can be detected by Raman scattering. One of the at least one planar alkyne is generated from the cyclopropenone of Formula I. Cyclopropenone of Formula I undergoes photolysis upon irradiation with light resulting in alkyne formation and carbon monoxide release.

In some embodiments, the photoactivatable vibrational probe can be converted to a vibrational probe having the following structure:

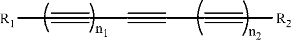

wherein R₁ and R₂ independently represent a monocyclic or polycyclic, aromatic or heteroaromatic ring; n₁ and n₂ each independently is 0 or 1.

In some embodiments, the photoactivatable vibrational probe can be converted to a vibrational probe having the following structure:

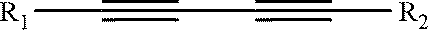

wherein R₁ and R₂ independently represent a monocyclic or polycyclic, aromatic or heteroaromatic ring. In some embodiments, one or both of R₁ and R₂ is a phenyl group.

In some embodiments, the photoactivatable vibrational probe can be converted to a vibrational probe having the following structure:

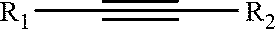

wherein R₁ and R₂ independently represent a monocyclic or polycyclic, aromatic or heteroaromatic ring. In some embodiments, one or both of R₁ and R₂ is a phenyl group. The phenyl group can be substituted or unsubstituted.

In some embodiments, the photoactivatable vibrational probe described herein has a structure according to Formula III:

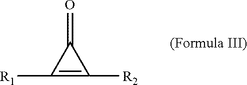

wherein R₁ and R₂ independently represent a monocyclic or polycyclic, aromatic or heteroaromatic ring. In some embodiments, one or both of R₁ and R₂ is a phenyl group.

In some embodiments, the photoactivatable vibrational probe described herein has a structure according to Formula III:

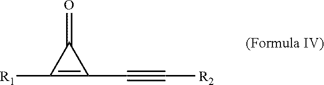

wherein R₁ and R₂ independently represent a monocyclic or polycyclic, aromatic or heteroaromatic ring. In some embodiments, one or both of R₁ and R₂ is a phenyl group.

In some embodiments, the photoactivatable vibrational probe described herein has a structure having the following Formula:

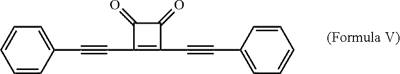 [SMILES: O=c1c(C#Cc2ccccc2)c(C#Cc2ccccc2)c1=O]

In some embodiments, the photoactivatable vibrational probe described herein has a formula of:

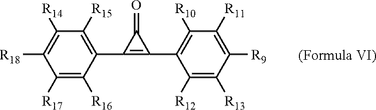

wherein R₉, R₁₀, R₁₁, R₁₂, R₁₃, R₁₄, R₁₅, R₁₆, R₁₇, and R₁₈ can each independently represent a hydrogen or a steric group including alkyl (e.g., C1-C6 alkyl), aryl, heteroaryl, halogen, hydroxyl, alkyl alcohol, carboxyl, alkoxyl, aryloxyl, thio, alkythio, amino, and alkylamino, aldehyde, alkenyl, alkynyl, benzyl, carboxamide, azo, ester, carbonyl, nitrile, nitro, phenyl, sulfonyl, sulfinyl. In some embodiments, at least one of R₉, R₁₀, R₁₁, R₁₂, R₁₃, R₁₄, R₁₅, R₁₆, R₁₇, and R₁₈ is an alkyl group (e.g., a methyl group). In some embodiments, at least one of R₉, R₁₀, R₁₁, R₁₂, and R₁₃ is an alkyl group (e.g., a methyl group). In some embodiments, at least one of R₁₄, R₁₅, R₁₆, R₁₇, and R₁₈ is an alkyl group (e.g., a methyl group). In some embodiments, at least one of R₁₀ and R₁₂ is an alkyl group and at least one of R₁₅ and R₁₆ is an alkyl group. In some embodiments, R₉, R₁₀, and R₁₂ can be an alkyl group, and R₁₁ and R₁₃ can be H. In some embodiments, R₁₅, R₁₆, and R₁₈ can be an alkyl group, and R₁₄ and R₁₇ can be H.

In some embodiments, the photoactivatable vibrational probe described herein has a formula of:

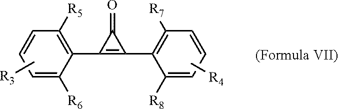

wherein R₃ and R₄ are independently a hydrogen, a hydroxyl group, an alkyl group, an alkoxy group, an amide, a ketone, a carboxyl group, a halogen, an ether, or an ester. R₅, R₆, R₇ and R₈ are independently hydrogen or steric groups including alkyl, aryl, heteroaryl, halogen, hydroxyl, alkyl alcohol, carboxyl, alkoxyl, aryloxyl, thio, alkythio, amino, and alkylamino, aldehyde, alkenyl, alkynyl, benzyl, carboxamide, azo, ester, carbonyl, nitrile, nitro, phenyl, sulfonyl, sulfinyl.

In some embodiments, the photoactivatable vibrational probe described herein has a formula of:

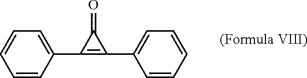 [SMILES: O=c1c(-c2ccccc2)c1-c1ccccc1]

In some embodiments, the photoactivatable vibrational probe described herein has a formula of:

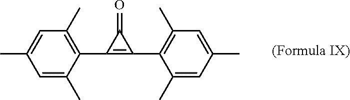

In some embodiments, incorporating one or more alkyl groups around cyclopropenones of a photoactivatable vibrational probe disclosed herein can shield the strained ketones from nucleophilic attack and improve the stability of the photoactivatable vibrational probe inside cells. In some embodiments, alkyl shielded (e.g., methyl shielded) photoactivatable vibrational probe exhibits improved stability to nucleophiles and enhanced uncaging Raman signals. In some embodiments, incorporating one or more methyl groups around cyclopropenones of the photoactivatable vibrational probe can present a Raman peak shift from unmethylated cyclopropenone.

In some embodiments, at least one of the R₉, R₁₀, R₁₁, R₁₂, R₁₃, R₁₄, R₁₅, R₁₆, R₁₇, and R₁₈ in Formula VI can be attached to a targeting moiety. Similarly, at least one of R₃, R₄, R₅, R₆, R₇ and R₈ in Formula VII can be attached to a targeting moiety. In some preferred embodiments, at least one of R₃ and R₄ can be attached to a targeting moiety. The attachment can be directly or indirectly via a chemical handle (e.g., phenol alcohol handle). For example, in some embodiments, one of the hydrogen in one or both of the phenyl group of Formula VII and VIII or one of the methyl groups in one or both of the phenyl group of Formula IX can be substituted with a hydroxyl group. A targeting moiety can be covalently attached to the photoactivatable vibrational probe via the hydroxyl group.

A targeting moiety as used herein refers to a chemical moiety that can recognize and bind to a receptor on a target such as an organelle in a cell or a specific cell type. Typically, the binding of a targeting moiety to a receptor on a target is a high affinity binding interaction. A targeting moiety can be a small molecule, a nucleic acid, a polypeptide, glycopeptide, proteoglycan, carbohydrate, lipid, or others identifiable to a person skilled in the art.

The target moiety attached to the photoactivatable vibrational probe can target a cell or a component of a cell such as organelles and biomolecules including nucleic acids, lipids, sugars, proteins and metabolites. In some embodiments, a targeting moiety can recognize one or more receptors or targets or markers associated with a particular organelle, cell component, cell, tissue or organ. In some embodiments, a target molecule can be a cell marker that is exclusively or primarily associated with a specific cell type, a disease, and/or a developmental stage. For example, a targeting moiety can be a nucleic acid targeting moiety that binds to a cell type specific marker. In some embodiments, a targeting moiety can be a naturally occurring or synthetic ligand for a cell surface protein. In some embodiments, the targeting moiety can be an organelle targeting moiety that targets an organelle of a cell such as lipid droplet, mitochondria, Golgi, nucleus, endoplasmic reticulum, or liposome. Any organelle targeting moiety described herein and known in the art can be employed herein. In some embodiments, targeting to cell organelles can occur through receptor-mediated endocytosis. A targeting moiety can be a basic group (e.g., a dimethylamine group) for acidic organelle targeting. In some embodiments, the targeting moiety mediated delivery of the probes is based on membrane potential created by high negative potential of an organelle (e.g., mitochondria). For example, in some embodiments, a targeting moiety can be a lipophilic cation for mitochondria targeting, such as an alkyltriphenylphosphonium moiety (e.g., positively charged triphenylphosphonium (TPP)). The attachment of TPP to a photoactivatable vibrational probe delivers the probe initially to the cytoplasm and subsequently to mitochondria. In some embodiments, a targeting moiety is a methyl sulphonamide group. In some embodiments, a targeting moiety is a halotag ligand. In some embodiments, an organelle targeting moiety can be a nuclear localization signal, Tat peptide or mitochondrial targeting sequence.

In some embodiments, the photoactivatable vibrational probe described herein has a formula of:

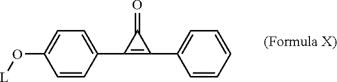

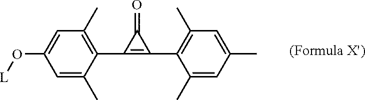

wherein L represents a targeting moiety.

In some embodiments, the photoactivatable vibrational probe has one of the following structures:

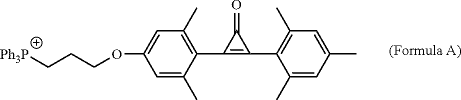

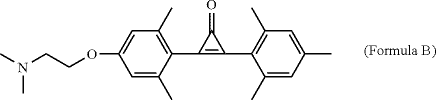

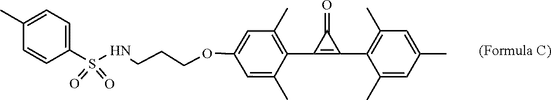

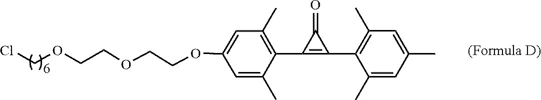

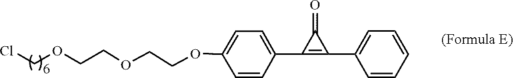

In some embodiments, a photoactivatable vibrational probe described herein can be isotopically modified. For example, upon photoactivation, the photoactivatable vibrational probe can generate a vibrational probe comprising an isotopically modified alkyne(s). In some embodiments, the photoactivatable vibrational probe comprises at least one ¹³C atom. For example, the photoactivatable vibrational probe can comprise one, two or more ¹³C atom.

In some embodiments, the photoactivatable vibrational probe has a formula of:

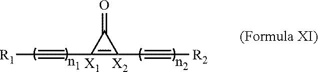

wherein one or both of X₁ and X₂ is a ¹³C atom; R₁ and R₂ independently represent a monocyclic or polycyclic, aromatic or heteroaromatic ring; and n₁ and n₂ are independently 0 or 1. In some embodiments, one or both of R₁ and R₂ is a phenyl group.

In some embodiments, the photoactivatable vibrational probe has a formula of:

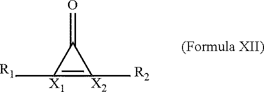

wherein one or both of X₁ and X₂ is a ¹³C atom and R₁ and R₂ independently represent a monocyclic or polycyclic, aromatic or heteroaromatic ring. In some embodiments, one or both of R₁ and R₂ is a phenyl group. Accordingly, in some embodiments, the photoactivatable vibrational probe can have a structure of Formula XIII:

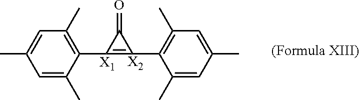

wherein one or both of X₁ and X₂ is a ¹³C atom and one of the methyl group in one or both phenyl group can be substituted with a targeting moiety. For example, the photoactivatable vibrational probe can have a structure of Formula XIV:

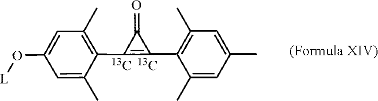

The photoactivatable vibrational probes described herein can enable photoactivatable Raman imaging while preserving multiplexity and small-size features. In some embodiments, the photoactivatable vibrational probes are small molecules, therefore introducing much less physical perturbation when tagging biomolecules. As used herein, the term “small molecules” refers to low molecular weight organic compound having a molecular weight of 1000 Daltons or less. In some embodiments, the small molecules have a size on the order of 10⁻⁹ m.

In some embodiments, the photoactivatable vibrational probe is biocompatible with living cells, living tissues/organs, or with a subject (e.g., human). The term “biocompatible” as used herein in the present disclosure can refer to molecules or compounds that are generally non-toxic and cause no significant adverse effects to the recipient. For example, a biocompatible material is a material that does not elicit a significant inflammatory or immune response when administered to a subject. In some embodiments, a biocompatible material elicits no detectable change in one or more biomarkers indicative of an immune response.

In some embodiments, the photoactivatable vibrational probes herein described exhibit multiple distinct Raman peaks within the cell-silent Raman window (1800 - 2800 cm⁻¹), therefore allowing multiple color imaging with Raman scattering. In some embodiments, the photoactivatable vibrational probe can exhibit a narrow spectral width for a specific detection which can reduce the probability of overlapping with other probes. Raman scattering used herein can be spontaneous Raman scattering or stimulated Raman scattering. The photoactivatable vibrational probes herein described demonstrate in some embodiments superior quantitative imaging capabilities without the complications of photobleaching and the need of post-processing linear unmixing. In some embodiments, the photoactivatable vibrational probes described herein can be used for multiplexed imaging and tracking as well as for monitoring interactions between cells and/or cell components with substantially enhanced specificity, sensitivity and multiplex capability in vitro, ex vivo (e.g., live cell or tissue) or in vivo.

### Imaging With Photoactivatable Vibrational Probes

Provided herein also includes a method for imaging a biological material using the photoactivatable vibrational probes described herein. In some embodiments, the method comprises contacting at least one photoactivatable vibrational probe described herein with the biological material. The at least one photoactivatable vibrational probe is provided at a detectably effective amount sufficient to yield an acceptable image using Raman scattering (e.g., SRS imaging). The method can also comprise activating the at least one photoactivatable vibrational probe with light (e.g., UV light) to generate at least one vibrational probe and detecting the at least one vibrational probe in the biological material using Raman scattering.

A biological material can include a material derived from, obtained from, or located in a biological subject (e.g., a mammal). In some embodiments, a biological material can be a material in vitro, in situ or in vivo. Examples of the biological material include a biological tissue or fluid or a fraction thereof, an organ, a cell (e.g., live cell), living organism, a cell line, a cell constituent derived from or located in mammals including humans. In some embodiments, the biological material comprises live cells or organism. In some embodiments, the biological material can include a collection of cells obtained from, derived from or in a tissue of the subject such as, for example, epithelium, connective tissue, blood vessels, muscle, nerve tissue, bone from any time in development of the subject. In some embodiments, the tissue can include live animal cells. In some embodiments, the biological material includes healthy, diseased, or malignant tissue (e.g., cancerous or tumor tissue). A biological material can be a fluid such as urine, serum, blood plasma, or blood. In some embodiments, a biological material comprises a tissue. A tissue can be a connective tissue (e.g., blood and lymph tissue), a nervous tissue (e.g., central nervous system (CNS) or peripheral nervous system (PNS)), an epithelial tissue (e.g., skin tissue) or a muscle tissue. In some embodiments, a biological material comprises a tissue section for use in Raman imaging application or fluorescence or fluorescence-related imaging applications.

Contacting at least one photoactivatable vibrational probe with the biological material can occur in vivo, ex vivo, in vitro, or a combination thereof for a suitable time period allowing the photoactivatable vibrational probe to interact with the target in the biological material (e.g., receptor molecules on a cell component). The time period can vary in different embodiments. For example, in some embodiments, the time period can be 5 minutes, 10 minutes, 15 minutes, 30 minutes, 1 hour, 2 hours, 4 hours, 8 hours, 12 hours, 16 hours, 20 hour, 24 hours or longer.

In some embodiments, a photoactivatable vibrational probe used herein has a structure according to any one of the formulas described herein. For example, a photoactivatable vibrational probe can have a structure according to Formula I, II, III, VI, V, VI, VII, VIII, IX, X, X′, XI, XII, XIII or XIV. The photoactivatable vibrational probe can further comprise a targeting moiety described herein. The targeting moiety can be directly or indirectly attached to the photoactivatable vibrational probe via a chemical handle.

In some embodiments, two or more photoactivatable vibrational probes (e.g., two, three, four or more) are introduced to the biological material. The two or more photoactivatable vibrational probes, when activated, can exhibit different Raman peaks. In some embodiments, the Raman peaks exhibited by the two or more photoactivatable vibrational probes are distinguishable from one another with minimally overlapping spectrum. In some embodiments, at least one of the two or more photoactivatable vibrational probes is isotopically modified. For example, the photoactivatable vibrational probe can comprise one or more ¹³C atoms. In some embodiments, none of the two or more photoactivatable vibrational probes is isotopically modified.

In some embodiments, one of the photoactivatable vibrational probes can have a structure according to Formula I and another photoactivatable vibrational probe can have a same structure with one, two or more isotope substitutions such as in Formula XI. In some embodiments, one of the photoactivatable vibrational probes can have a structure according to Formula III and another photoactivatable vibrational probe can have a same structure with one, two or more isotope substitutions such as in Formula XII. In some embodiments, one of the two or more photoactivatable vibrational probes can have a structure of Formula IX (or with one of the methyl group substituted with a targeting moiety), and another of the two or more photoactivatable vibrational probes can have a structure according to Formula XIII, such as the structure of Formula XIV.

In some embodiments, the two or more photoactivatable vibrational probes have a structure according to Formula VI with different R groups. The two or more photoactivatable vibrational probes can have different number of alkyl groups in one or both phenyl groups of Formula VII. For example, one of the photoactivatable vibrational probes can have no alkyl group in the phenyl groups (e.g., a structure of Formula VIII), and another photoactivatable vibrational probe can have one or more methyl groups in one or both phenyl groups (e.g., methyl shielded as in Formula IX). In some embodiments, two photoactivatable vibrational probes are introduced to the biological material, one having a structure of Formula X and the other having a structure of Formula X′. In some embodiments, three photoactivatable vibrational probes are introduced to the biological material, a first probe having a structure of Formula X, a second probe having a structure of Formula X′, and a third probe having a structure of Formula XIV.

The photoactivatable vibration probe used herein can further comprise a targeting moiety described herein. The targeting moiety can target a specific cell organelle, cellular component such as proteins, lipids or nucleic acids, a specific cell type, or cells at a developmental stage. In some embodiments, the two or more photoactivatable vibration probes introduced to a biological material each comprises a targeting moiety different from one another, thereby delivering the two or more photoactivatable vibration probes to different targets (e.g., different biomolecules, different cell organelle, different cell types, or different tissues or organs) in the biological materials. In some embodiments, the targeting moieties can target cell organelles such as mitochondria, Golgi, nucleus, lysosome, endoplasmic reticulum or lipid droplets by interacting with the receptor or molecules associated with those organelles.

In some embodiments, two or more photoactivatable vibrational probes are introduced to the biological materials, each having a structure selected from Formulas A-E. For example, the two or more photoactivatable vibrational probes can each have a structure selected from Formula A-D. Alternatively or in addition, the two or more photoactivatable vibrational probes can each have a structure selected from Formula A-D and E. The photoactivatable vibrational probes can be further isotopically modified. Accordingly, in some embodiments, the photoactivatable vibrational probes can have a structure of:

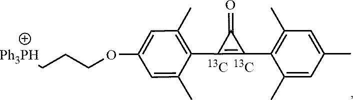

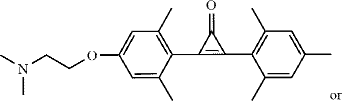

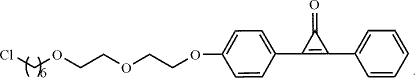

The photoactivatable vibrational probe introduced to the biological material can be activated by applying light (laser or non-laser) to the biological material, thereby converting the photoactivatable vibrational probe into a vibrational probe that can provide a Raman signal in the cell-silent Raman window. The light can be provided by a laser light source or a non-laser light source. In some embodiments, activating the photoactivatable vibrational probe comprises irradiating the photoactivatable vibrational probe with light having a wavelength selectively absorbed by the cyclopropenone. In some embodiments, the conversion of a photoactivatable vibrational probe to a vibrational probe for detection by Raman scatting can be induced through one-photon or multi-photon absorption using a light source covering wavelength across UV to near infrared light from about 200 nm to about 1500 nm. In some embodiments, the light can have a wavelength from about 220 nm to about 450 nm, from about 325 nm to about 375 nm, from about 325 nm to about 355 nm, and from about 350 nm to about 355 nm. The alkyne Raman signal generated from photoactivation can then be detected with Raman scattering.

The method can further comprise detecting the at least one vibrational probe in the biological material with Raman scattering imaging. The Raman scattering can be spontaneous Raman scattering or stimulated Raman scattering. In some embodiments, the Raman scattering is stimulated Raman scattering (SRS). As will be understood by a skilled person, SRS directs two monochromatic laser beams, a pump laser beam and a Stokes laser beam, on a sample. When the difference in frequency between both photons resembles that of a specific vibrational transition, the occurrence of this transition is resonantly enhanced. The SRS signal is equivalent to changes in the intensity of the pump and Stokes beams.

Accordingly, the at least one vibrational probe in the biological material can be detected using a device comprising a first laser generator that can produce a pulse laser beam of a first fixed wavelength (e.g., a pump beam), a second laser generator that can produce a pulse laser beam at a second fixed wavelength (e.g., a Stokes beam), a modulator that can modulate the pulse laser beam of the first or second laser generator, a photodetector that can be adapted to detect the stimulated Raman scattering from the biological material, and a computer that generates images of the vibrational probes (e.g., alkynes) based on the detected stimulated Raman scattering. The first and second laser generators can be configured to provide a pump radiation and a Stokes radiation, each at a fixed wavelength whose energy difference can be between, for example, about 2000 and 2500 wavenumbers. In some embodiments, a laser of the SRS device can be tuned to a particular frequency based on the bond to be detected in the vibrational probe (e.g., alkynes or isotopically modified alkynes). In some embodiments, the energy difference between the photons produced by the first laser radiation and the photons produced by the second laser radiation matches with the energy of the vibrational transitions of alkynes or isotopically modified alkynes generated from the cyclopropenone of Formula I. In some embodiments, the vibrational probes can also be detected by spontaneous Raman micro-spectroscope (e.g., an upright confocal Raman spectrometer (Horiba Raman microscope; Xplora plus) equipped with a 532 nm or 785 nm or 1064 nm laser). Spectro/Raman shift center can be set to be 2000.04 cm⁻¹. With a 1200 grating (750 nm), Raman shift ranges from 690.81 to 3141.49 cm⁻¹ can be acquired to cover whole cellular Raman peaks.

In some embodiments, the methods described herein can be used to monitor or track the locations of a target cell or cellular component, the interactions of two or more cells or cellular components, or to trace a cellular process in a live cell. Following the photoactivation of a photoactivatable vibrational probe in a biological material, the physical movement, the chemical reaction or the biological interactions of two or more components labeled by the photoactivatable vibrational probe can be monitored within the cell by Raman scattering. In some embodiments the cellular process can be a DNA replication, RNA synthesis, protein synthesis, protein degradation, glucose uptake or drug uptake.

Accordingly, in some embodiments, the method can comprise contacting at least one photoactivatable vibrational probe with a biological material, activating the at least one photoactivatable vibrational probe with light to generate at least one vibrational probe, and detecting the at least one vibrational probe in the biological material by Raman scattering at a first time point. The method can further comprise detecting the at least on vibrational probe biological material by Raman scattering at a second time point and comparing images obtained at the two time points. The time interval between the first time point and the second time point can vary in different embodiments. In some embodiments, the time interval between the first time point and the second time point can be about 1 minute, 2 minutes, 5 minutes, 10 minutes, 15 minutes, 20 minutes, 25 minutes, 30 minutes, 40 minutes, 50 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, 24 hours or two or more days. In some embodiments, the detecting can be performed at a series of time points with any suitable time interval, generating a series of images.

The photoactivation of the cyclopropenone in Formula I also releases carbon monoxide in addition to the vibrational probe. Accordingly, in some embodiments, a method for controlled releasing of carbon monoxide (CO) into a biological material is also described. The method can comprise introducing a photoactivatable vibrational probe described herein to the biological material and activating the photoactivatable vibrational probe with light, thereby releasing CO into the biological material. The CO release can be controlled by, for example, time duration of the illumination. The CO released into the biological material can be detected by any suitable imaging procedure known in the art, such as infrared imaging. The amount of CO released into the biological material can also be spatially and temporally quantified using the co-released (activated) alkynes by Raman imaging described above. The caged CO carrier (cyclopropenones) can be spatially and temporally tracked by infrared or Raman imaging for their vibrational peaks (~ 1850 cm⁻¹) in the cell silent region. In some embodiments, the biological material can be imaged (e.g., using Raman spectroscopy or infrared imaging) prior to the photoactivation, after the photoactivation, and/or during the course of the photoactivation to directly or indirectly monitor the potential location CO can be released to. For example, the CO released to the biological material can be directly detected by infrared imaging during the course of the photoactivation and/or after the photoactivation. In some embodiments, the uncaged vibrational probes in the biological material can be detected by Raman scattering during the course of the photoactivation and/or after the photoactivation to indirectly monitor the release of CO (e.g., location of the release).

Provided herein also includes a method of imaging a subject. In some embodiments, the method comprises administering the composition comprising at least one photoactivatable vibrational probe at a detectably effective amount to a subject, activating the at least one photoactivatable vibrational probe with light to generate at least one vibrational probe, and imaging the subject using Raman scattering.

Accordingly, provided herein also includes a composition for Raman imaging. The composition can comprise at least one photoactivatable vibrational probe described herein. The composition can further comprise appropriate buffering agents, reagents or carriers known in the art suitable for administration to a subject.

As used herein, the term “detectably effective amount” refers to an amount that, when introduced into a subject, is sufficient to yield an acceptable image using Raman imaging (e.g., SRS imaging). The effective amount of the probe can vary according to the type of photoactivatable vibrational probes, the subject that the photoactivatable vibrational probe is introduced into, instrument and digital processing related factors as will be understood by a person skilled in the art. In some embodiments, a “detectably effective amount” is the amount that is sufficient to reach an in vivo concentration of 1 µM to 100 mM, 3 µM to 30 mM, 10 µM to 10 mM, 100 µM to 1 mM, 10 µM to 1 mM or 10 µM to 100 mM in a target cell or organ. In some embodiments, a detectably effective amount of the probe can be administered in more than one injection.

Administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, and/or intradermal injection, or any combination thereof.

In methods described herein, the probe composition can be administered to a subject in any way suitable to deliver the photoactivatable vibrational probe to a target site. In some embodiments, the composition can be administered to the target site locally or systematically.

The term “local administration” or “topic administration” as used herein indicates any route of administration by which a composition is brought in contact with the body of the individual, so that the resulting composition location in the body is topic (limited to a specific tissue, organ or other body part where the imaging is desired). Exemplary local administration routes include injection into a particular tissue by a needle, gavage into the gastrointestinal tract, and spreading a solution containing hydrogel composition on a skin surface.

The term “systemic administration” as used herein indicates any route of administration by which a composition is brought in contact with the body of the individual, so that the resulting composition location in the body is systemic (i.e. non limited to a specific tissue, organ or other body part where the imaging is desired). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion. In some embodiments, the administration is systemic. In some embodiments, the administration is intravenous.

In some embodiments, the subject has a disease or disorder or is suspected having a disease or disorder. The method described herein can be used for detecting a disease condition in a subject. For example, the method can comprise administering to the subject a composition comprising at least one photoactivatable vibrational probe having a targeting moiety targeting a disease tissue or pathogen. The method can further comprise activating the at least one photoactivatable vibrational probe with light to generate at least one vibrational probe and imaging the subject using Raman scattering. The disease condition can include cancer, metabolic syndrome (e.g., CDG syndrome), neurodegenerative diseases (e.g., Alzheimer’s, Parkinson’s, or Huntington’s), inflammatory diseases and microbial infection (e.g., bacterial, viral, or fungal infection).

In some embodiments, the disease condition includes diseases associated with cell organelles. Examples of diseases related to cell organelles include, but are not limited to, Alzheimer’s, CDG syndrome, progeria, ED muscular dystrophy, cardiomyopathy, cancer, diabetes mellitus and deafness, familial hypercholesterolemia, infectious diseases (e.g., HIV, shigella etc.), lysosomal storage disease (Tay Sachs disease), autoimmune diseases, cystic fibrosis, ER storage diseases, and others identifiable to a person skilled in the art.

The method described herein can, for example, be used to monitor a treatment of a disease condition in a subject. The method can comprise administering to the subject a composition comprising at least one photoactivatable vibrational probe, activating the at least one photoactivatable vibrational probe with light to generate at least one vibrational probe, and imaging the subject using Raman scattering at a first time point. The method can further comprise administering to the subject at least one photoactivable vibration probe, activating the at least one photoactivatable vibrational probe with light to generate at least one vibrational probe, and imaging the subject using Raman scattering at a second time point. The method further comprises comparing images obtained at the two time points. The first time point can be a time point about or prior to the initiation of the treatment and the second time point can be a time point after the initiation of the treatment. The first time and the second time point can be any two time points during the course of the treatment. The treatment is a treatment of any disease condition described herein. For example, the treatment is a treatment for cancer. In some exemplary embodiments, the treatment is a treatment for a disease condition associated with cell organelles.

### Kits

Provided herein also includes a kit for Raman imaging. A kit can comprise one or more photoactivatable vibrational probe described herein. The kit can further include appropriate buffering agents, media and carriers known in the art for administering the photoactivatable vibrational probe to a subject. In some embodiments, the kit may optionally contain a sterile and physiologically acceptable medium such as water, saline, buffered saline, and the like. A kit may comprise one or more unit doses described herein. The compositions can be in the form of kits of parts. In a kit of parts, one or more components of the compositions disclosed herein can be provided independent of one another and then employed (e.g., by a user) to generate the compositions.

The kit can further include instructions for using the components of the kits to practice the methods described herein such as to image live cells, cell organelles, or a subject. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g., CD-ROM, diskette, flash drive, etc. In some instances, the actual instructions are not present in the kit, but means for obtaining the instructions from a remote source (e.g., via the Internet), can be provided. An example of this embodiment is a kit that includes a web address where the instructions can be viewed and/or from which the instructions can be downloaded. As with the instructions, this means for obtaining the instructions can be recorded on a suitable substrate.

### EXAMPLES

Some aspects of the embodiments discussed above are disclosed in further detail in the following examples, which are not in any way intended to limit the scope of the present disclosure.

### Example 1

### General Synthesis Experimental Details

This example describes details of general synthesis experimental procedures, characterization methods and synthetic pathways used in the embodiments described in the present disclosure.

Reagents and solvents from commercial sources were used without further purification unless otherwise stated. All reactions were performed under a N2 atmosphere unless specified otherwise. All reaction flasks were flame dried. Column chromatography was carried out using SiliaFlash irregular silica gel P60 (Silicycle, 40 - 63 µm, 60 Å). Thin layer chromatography (TLC) was carried out with Millipore silica gel F-254 plates, and plates were visualized using UV light or KMnO4 stain. The UV handlamp is an Analytik Jena UVP EL Series Lamp (UVLS-24, 4 Watt, 2 UV 254/365 nm).

NMR spectra were recorded using a 400 MHz Bruker Avance III HD with Prodigy Cryoprobe or a 400 MHz Bruker Avance Neo. All ¹H NMR spectra are reported in δ units, parts per million (ppm), and were measured relative to the signals for CH2Cl2 (5.32 ppm) in deuterated solvent. All ¹³C NMR spectra were measured in deuterated solvents and are reported in ppm relative to the signals for 13CD2Cl2 (54.00 ppm). Multiplicity and qualifier abbreviations are as follows: s = singlet, d = doublet, t = triplet, q = quartet, m = multiplet, br = broad.

High resolution mass spectra (HRMS) were obtained from an LTQ linear ion trap mass spectrometer with liquid-chromatography (LC) system (Thermo) or LCT Premier XE electrospray TOF mass spectrometer with UPLC (Waters).

Preparative HPLC was performed on an Agilent 1100 Series semi-preparative HPLC apparatus using Eclipse XDB-C18 9.4 x 250 mm column with water (0.1% AcOH) and acetonitrile as mobile phases. UV-Vis absorption spectra were recorded on a Varian Cary 50 UV-Vis Spectrophotometer (Agilent). Analytical HPLC coupled with mass spectrometry (LC-MS) was performed on Agilent 1290 infinity LC system using ZORBAX RRHD Eclipse Plus C18, 95 Å, 2.1 x 50 mm, 1.8 µm column with an Agilent 6140 Series Quadrupole LCMS / LC-MS / MSD / Mass Spectrometer System. The mobile phase is water (0.1% AcOH) and acetonitrile with running method of gradient 5% - 95% acetonitrile (1 ml/min, 0-4 min). Mass spectrometry detection region ranges from 100 to 800 AMU.

### 2-phenyl-3-(phenylethynyl)cycloprop-2-en-1-one (2-cyclo, 3)

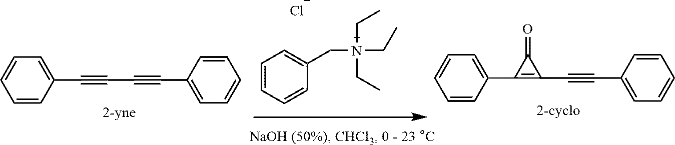

The synthesis procedure of 2-cyclo (3) was adapted from literature report. To a vigorously stirred mixture of 2-yne (4, 202 mg, 1 mmol) and benzyltriethylammonium chloride (114 mg, 0.5 mmol) in chloroform (8 mL), which had been cooled with an ice bath, was added 10 g of 50% aq. NaOH (w/w, 125 mmol) dropwise. Afterwards, the ice bath was removed and the solution was stirred for 40 minutes at 23° C. The reaction was monitored by TLC (1:3, EtOAc : Hexane). The resulting mixture was then separated, and the aqueous layer extracted with DCM. The combined organic layers were washed with saturated NaHCO3 and brine, dried over anhydrous Na2SO4, and evaporated. The crude product was purified by flash column chromatography with EtOAc : Hexane (1:5) as eluents. 2-cyclo (4) was isolated as yellow solid (74 mg, 0.32 mmol, 32%) and unreacted 2-yne (3) was also recovered (91 mg, 0.45 mmol, 45%).

¹H NMR (400 MHz, CD₂Cl₂) δ: 7.93 (m, 2H), 7.62 (m, 5H), 7.47 (m, 3H). ¹³C{¹H} NMR (100 MHz, CD₂Cl₂) δ 153.02, 151.18, 137.32, 133.90, 133.25, 131.53, 131.44, 129.89, 129.31, 124.30, 121.25, 108.77, 74.32. HRMS (FAB⁺, m/z): calcd. for [C17H11O]⁺ (M+H)⁺, 231.0810; found, 231.0838.

### (4-Oxo-2-phenylcycloprop-1-en-1-yl)phenoxy)propyl)triphenylphosphonium bromide (Cyclo-Mito, 7) and 2-(4-(2-(2-((6-chlorohexyl)oxy)ethoxy)ethoxy)phenyl)-3- phenylcycloprop-2-en-1-one (Cyclo-LD, 17)

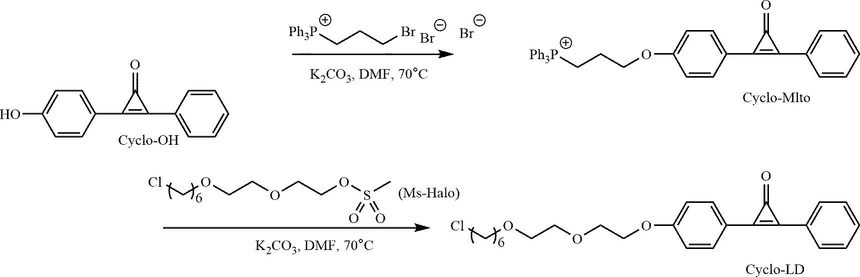

Cyclo-OH and Ms-Halo were produced according to literature. Cyclo-OH (22 mg, 0.1 mmol), K₂CO₃ (28 mg, 0.2 mmol) and (3-Bromopropyl)triphenylphosphonium bromide (139 mg, 0.3 mmol) or Ms-Halo (45 mg, 0.15 mmol) were stirred in 3 ml DMF at 70° C. for 1 hour. Then the reaction solution was filtered and quenched with saturated NH₄Cl solution. The solution was extracted by EtOAc and concentrated. The crude products were dissolved in acetonitrile and subjected to reverse phase prep-HPLC for separation to obtain Cyclo-Mito (7, 36 mg, 0.059 mmol, 59%) and Cyclo-LD (17, 29 mg, 0.068 mmol, 68%) as pale white solids. The products would decompose to Cyclo-OH on silica gel.

Cyclo-Mito (7). ¹H NMR (400 MHz, CD₂Cl₂) δ 7.95 - 7.78 (m, 13H), 7.70 (td, J = 8.0, 3.4 Hz, 6 H), 7.57 (ddt, J = 5.7, 4.0, 2.3 Hz, 3 H), 7.15 - 7.05 (m, 2 H), 4.42 (t, J = 5.8 Hz, 2 H), 4.08 - 3.87 (m, 2 H), 2.20 (m, 2 H)

HRMS (ESI, m/z): calcd. for [C₃₆H₃₀O₂P]⁺ (M⁺), 525.1978; found, 525.1960.

Cyclo-LD (17). 1 H NMR (400 MHz, CD₂Cl₂) δ 8.01 - 7.90 (m, 4 H), 7.59 (dp, J = 6.2, 2.0 Hz, 3 H), 7.17 - 7.05 (m, 2 H), 4.29 - 4.17 (m, 2 H), 3.89 - 3.82 (m, 2 H), 3.70 - 3.64 (m, 2 H), 3.61 - 3.49 (m, 4 H), 3.44 (t, J = 6.6 Hz, 2 H), 1.86 - 1.71 (m, 2 H), 1.62 - 1.51 (m, 2 H), 1.50 -1.29 (m, 4 H). ¹³C{¹H} NMR (100 MHz, CD₂Cl₂) δ 162.78, 155.47, 148.26, 145.02, 134.14, 132.55, 131.52, 129.70, 124.82, 117.43, 115.68, 71.54, 71.28, 70.52, 69.77, 68.35, 45.64, 33.00, 29.94, 27.09, 25.84.

HRMS (ESI, m/z): calcd. for [C25H30O4Cl]+ (M+H)+, 429.1833; found, 429.1845.

### 2-hydroxy-2,6-dimethylphenyl)-3-mesitylcycloprop-2-en-1-one (Me-1-cyclo-OH or MeOH or 8)

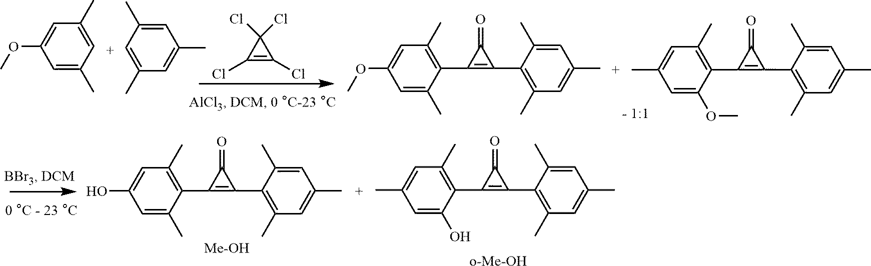

Me—OH (8) was produced following procedures modified from a previous report2 . In a flask, AlCl3 (1.49 g, 11.2 mmol) was charged to 10 ml dry DCM. The flask was then cooled to 0° C. To the stirring suspension, tetrachlorocyclopropene (500 mg, 2.8 mmol) was added dropwise. The resulting suspension was allowed to stir at 0° C. for 10 minutes. Then mesitylene (336 mg, 2.8 mmol) was added and this solution was stirred at 0° C. for 90 minutes. To this stirring suspension, 3,5-Dimethylanisole (381 mg, 2.8 mmol) was added. Then the solution was allowed to warm to 23° C. and reacted for 1 hour. This reaction was monitored by TLC (1:3, EtOAc : Hexane). After reaction, at 0° C. the suspension was quenched slowly with saturated NH₄Cl solution, extracted with DCM, washed with brine and dried over anhydrous Na2SO4. The organic phase was concentrated, and subjected to column chromatography with EtOAc : Hexane (1:5) as eluents. The obtained cyclopropenone products (703 mg, 2.3 mmol, 82%) were a mixture of ortho- and parasubstituted isomers (~ 1:1, identified by LC-MS).The two isomers were very close on TLC and column and hard to be separated. The isomers were separated at later step.

The obtained cyclopropenone isomers (306 mg, 1 mmol) were dissolved in 5 ml dry DCM in a flask. The flask was then cooled to 0° C. To the stirring suspension, 1 M BBr3 in DCM (2.5 ml, 2.5 mmol) was added dropwise. The stirred solution was kept at 0° C. for 1 hour, then allowed to warm to 23° C. and reacted overnight. After reaction, at 0° C. the suspension was quenched slowly with saturated NH₄Cl solution, extracted with DCM, washed with brine and dried over anhydrous Na2SO4. The organic phase was concentrated, and subjected to column chromatography with EtOAc : Hexane (1:2) as eluents to obtain Me—OH (7, 132 mg, 0.45 mmol) and o—Me—OH (120 mg, 0.41 mmol) as pale white solids. Note that these two isomers should be carefully separated on a long column, and these solid cyclopropenones have pleasant fragrance similar to that of banana shrub flowers.

Me—OH (8): ¹H NMR (400 MHz, CD₂Cl₂ with CD₃OD as co-solvent) δ 6.90 (d, J = 1.1 Hz, 2 H), 6.53 (s, 2 H), 4.31 (br, 1 H), 2.25-2.22 (s, 3 H), 2.21-2.18 (s, 6 H), 2.18-2.15 (s, 6 H). ^{l}³C{¹H} NMR (100 MHz, CD2Cl2 with CD3OD as co-solvent) δ 162.10, 160.00, 151.88, 148.49, 143.89, 142.59, 139.48, 129.32, 124.48, 117.37, 115.82, 21.30, 20.91, 20.50. HRMS (ESI, m/z): calcd. for [C₂₀H₂₁O₂]⁺ (M+H)⁺, 293.1537; found, 293.1539.

o—Me—OH— ¹H NMR (400 MHz, DMSO-d₆) δ 10.27 (br, 1 H), 6.99 (s, 2 H), 6.67 (s, 1 H), 6.62 (s, 1 H), 2.44 (s, 3 H), 2.33 - 2.23 (m, 12H). HRMS (ESI, m/z): calcd. for [C₃₆H₃₀O₂P]⁺ (M⁺), 293.1537; found, 293.1537.

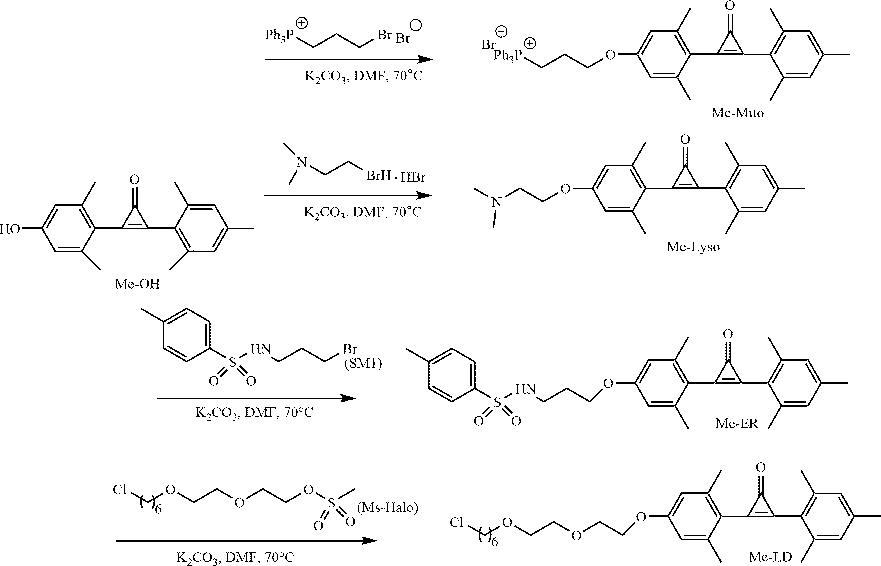

### (4-(2-Mesityl-3-oxocycloprop-1-en-1-yl)-3,5-dimethylphenoxy)propyl)triphenylphosphonium Bromide (Me-Mito, 12)

Me—OH (8, 29 mg, 0.1 mmol), K₂CO₃ (28 mg, 0.2 mmol) and (3-Bromopropyl)triphenylphosphonium bromide (139 mg, 0.3 mmol) were stirred in 3 ml DMF at 70° C. for 1 hour. Then the reaction solution was filtered and quenched with saturated NH₄Cl solution. The solution was extracted by EtOAc and concentrated. The crude product was subjected to column chromatography with (MeOH + AcOH) in DCM (10% + 2.5%) as eluents to obtain MeMito (12, 44 mg, 0.065 mmol, 65%) as pale white solid.

¹H NMR (400 MHz, CD₂Cl₂) δ 7.89 - 7.79 (m, 9 H), 7.75 - 7.68 (m, 6 H), 6.98 (s, 2 H), 6.70 (s, 2 H), 4.39 - 4.31 (m, 2 H), 3.95 (m, 2 H), 2.38 - 2.25 (m, 15 H), 2.22 - 2.15 (m, 2 H). ¹³C{¹H} NMR (100 MHz, CD₂Cl₂) δ 161.20, 158.11, 151.85, 150.56, 142.77, 142.05, 139.44, 135.71, 134.33, 134.23, 131.06, 130.94, 129.12, 124.59, 119.91, 119.04, 118.18, 114.44, 67.26, 67.09, 23.27, 21.67, 21.26, 20.83, 20.65, 20.12.

HRMS (ESI, m/z): calcd. for [C₄₁H₄₀O₂P]⁺ (M⁺), 595.2761; found, 595.2755.

### 2-(2-(dimethylamino)ethoxy)-2,6-dimethylphenyl)-3-mesitylcycloprop-2-en-1-one (MeLyso, 13)

Me—OH (8, 29 mg, 0.1 mmol), K2CO3 (41 mg, 0.3 mmol) and 2-bromo-N,N-dimethylethanamine hydrobromide (46 mg, 0.2 mmol) were stirred in 3 ml DMF at 70° C. for 1 hour. Then the reaction solution was filtered and quenched with saturated NH₄Cl solution. The solution was extracted by EtOAc and concentrated. The crude product was subjected to column chromatography with 7% MeOH in DCM as eluents to obtain Me-Lyso (13, 28 mg, 0.076 mmol, 76%) as pale white solid.

¹H NMR (400 MHz, CD₂Cl₂) δ 6.98 (s, 2 H), 6.70 (s, 2 H), 4.11 (t, J = 5.7 Hz, 2 H), 2.75 (t, J = 5.7 Hz, 2 H), 2.36 - 2.31 (m, 15H), 2.28 (s, 6 H). ¹³C{¹H} NMR (100 MHz, CD₂Cl₂) δ 161.84, 158.08, 152.04, 150.29, 142.82, 141.91, 139.36, 129.10, 124.77, 119.51, 114.39, 66.57, 58.53, 46.04, 21.67, 21.31, 20.83.

HRMS (ESI, m/z): calcd. for [C₂₄H₂₉NO₂]⁺ (M+H)⁺, 364.2272; found, 364.2279.

### N-(4-(2-mesityl-3-oxocycloprop-1-en-1-yl)-3,5-dimethylphenoxy)propyl)-4-methylbenzenesulfonamide (Me-ER, 14)

SM1 was produced according to literature. Me—OH (8, 29 mg, 0.1 mmol), K2CO3 (28 mg, 0.2 mmol) and SM1 (88 mg, 0.3 mmol) were stirred in 3 ml DMF at 70° C. for 1 hour. Then the reaction solution was filtered and quenched with saturated NH₄Cl solution. The solution was extracted by EtOAc and concentrated. The crude product was subjected to column chromatography with EtOAc : Hexane (1:1.5) as eluents to obtain Me—ER (14, 32 mg, 0.064 mmol, 64%) as pale white solid.

¹H NMR (400 MHz, CD2Cl2) δ 7.72 (d, J = 8.2 Hz, 2 H), 7.29 (d, J = 8.2 Hz, 2 H), 6.98 (s, 2 H), 6.63 (s, 2 H), 4.94 (t, J = 6.2 Hz, 1 H), 4.00 (t, J = 5.6 Hz, 2 H), 3.20 - 3.06 (m, 2 H), 2.40 (s, 3 H), 2.37 - 2.30 (m, 9 H), 2.28 (s, 6 H), 1.94 (p, J = 6.2 Hz, 2 H). ¹³C{¹H} NMR (100 MHz, CD₂Cl₂) δ 161.62, 158.12, 151.92, 150.39, 144.15, 142.78, 141.99, 139.38, 137.48, 130.24, 129.12, 127.53, 124.68, 119.63, 114.33, 65.97, 41.16, 29.64, 21.79, 21.67, 21.29, 20.84.

HRMS (ESI, m/z): calcd. for [C₃₀H₃₄NO₄S]⁺ (M+H)⁺, 504.2204; found, 504.2207

### 2-(2-(2-((6-Chlorohexyl)oxy)ethoxy)ethoxy)-2,6-dimethylphenyl)-3-mesitylcycloprop-2- En-1-one (Me-LD, 15)

Me—OH (8, 29 mg, 0.1 mmol), K₂CO₃ (41 mg, 0.3 mmol) and Ms-Halo (60 mg, 0.2 mmol) were stirred in 3 ml DMF at 70° C. for 4 hours. Then the reaction solution was filtered and quenched with saturated NH4Cl solution. The solution was extracted by EtOAc and concentrated. The crude product was subjected to column chromatography with EtOAc : Hexane (1:2) as eluents to obtain Me-LD (15, 35 mg, 0.070 mmol, 70%).

¹H NMR (400 MHz, CD2Cl2) δ 6.97 (s, 2 H), 6.71 (s, 2 H), 4.19 - 4.11 (m, 2 H), 3.87 - 3.78 (m, 2 H), 3.68 - 3.63 (m, 2 H), 3.59 - 3.50 (m, 4 H), 3.44 (t, J = 6.6 Hz, 2 H), 2.33 (d, J = 2.0 Hz, 9 H), 2.28 (s, 6 H), 1.76 (dq, J = 8.1, 6.7 Hz, 2 H), 1.60 - 1.53 (m, 2 H), 1.48 - 1.35 (m, 4 H). ¹³C{¹H} NMR (100 MHz, CD2Cl2) δ 161.83, 158.09, 152.01, 150.36, 142.80, 141.93, 139.38, 129.10, 124.74, 119.61, 114.42, 71.70, 71.39, 70.69, 70.03, 68.11, 45.80, 33.17, 30.10, 27.26, 26.00, 21.67, 21.31, 20.83.

HRMS (ESI, m/z): calcd. for [C₃₀H₄₀ClO₄]⁺ (M+H)⁺, 499.2610; found, 499.2620.

### (4-(2-mesityl-3-oxocycloprop-1-en-1-yl-1,2-13C2)-3,5-dimethylphenoxy)propyl)triphenylphosphonium Bromide (Me-Mito¹³C, 16)

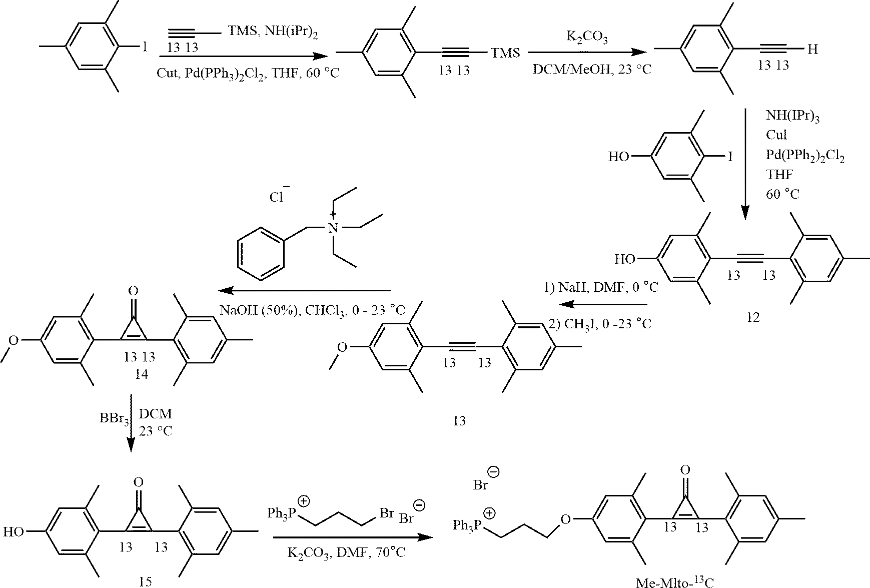

2-Iodo-1,3,5-trimethylbenzene (246 mg, 1 mmol), (trimethylsilyl)acetylene-¹³C₂ (Sigma 603511, 100 mg, 1 mmol), copper(I) iodide (10 mg, 0.05 mmol), bis(triphenylphosphine)palladium(II) dichloride (35 mg, 0.05 mmol) were dissolved in THF (5 ml). The mixture was degassed and filled with nitrogen. Then diisopropylamine (202 mg, 2 mmol) was added and the black suspension was heated to 60° C. and stirred for 2 hours. After the reaction was finished, the reaction solution was filtered and quenched with 1 M HCl. Then the solution was extracted by EtOAc and hexane. The organic phase was washed with brine, dried over anhydrous Na2SO4 and subjected to column chromatography with pure hexane as eluent to obtain I1 (157 mg, 0.72 mmol, 72%) as yellow oil.

I1. ¹H NMR (400 MHz, CD₂Cl₂) δ 6.87 (s, 2 H), 2.39 (s, 6 H), 2.28 (s, 3 H), 0.28 (dd, J = 2.3, 0.7 Hz, 9 H). ¹³ C{¹H} NMR (100 MHz, CD₂Cl₂) δ 141.01, 128.03, 104.46, 103.13, 102.72, 101.38, 21.65, 21.21, 0.48.

To a solution of I1 (109 mg, 0.5 mmol) in DCM-MeOH (1:1, 4 mL) was added K₂CO₃ (276 mg, 2 mmol). After the mixture was stirred at 23° C. for 2 hours, H₂O was added and the mixture was extracted with EtOAc. The organic phase was washed with brine, dried over anhydrous Na₂SO₄ to obtain the product without further purification.

The whole products obtained in the last step, 4-Iodo-2,6-dimethylphenol (124 mg, 0.5 mmol), copper(I) iodide (5 mg, 0.026 mmol), bis(triphenylphosphine)palladium(II) dichloride (18 mg, 0.025 mmol) were dissolved in THF (3 ml). The mixture was degassed and filled with nitrogen. Then diisopropylamine (101 mg, 1 mmol) was added and the black suspension was heated to 60° C. and stirred for 2 hours. After the reaction was finished, the reaction solution was filtered and quenched with 1 M HCl. Then the solution was extracted by EtOAc and hexane. The organic phase was washed with brine, dried over anhydrous Na₂SO₄ and subjected to column chromatography with EtOAc : hexane (1:2) as eluent to obtain I2 (98 mg, 0.37 mmol, 74%, over two steps) as white solids.

¹H NMR (400 MHz, CD₂Cl₂) δ 7.45 - 7.35 (m, 1 H), 6.92 (s, 2 H), 6.59 (s, 2 H), 2.48 (d, J = 3.6 Hz, 12 H), 2.29 (s, 3 H). ¹³C{¹H} NMR (100 MHz, CD2Cl2) δ 155.87, 142.56, 140.16, 137.91, 128.16, 114.47, 97.07, 95.30, 95.17, 93.40, 21.99, 21.84, 21.58.

I2 (98 mg, 0.37 mmol) was dissolved in DMF (2 ml) and cooled to 0° C. NaH (12 mg, 0.5 mmol) was added to the stirred solution. Stirring was maintained for 30 minutes at 0° C. Then the mixture was added iodomethane (105 mg, 0.74 mmol, dissolved in 1 ml DMF) and allowed to warm up to 23° C. and stirred for 2 hours. Then the mixture was poured over water and extracted with EtOAc. The organic phase was washed with brine, dried over anhydrous Na₂ SO₄ to obtain the product I3 without further purification.

All the product I3 obtained in the last step was dissolved in chloroform (4 mL) and added with benzyltriethylammonium chloride (68 mg, 0.3 mmol). Then the mixture was cooled with an ice bath, and was added 3 g of 50% NaOH (37.5 mmol) dropwise. After adding NaOH, ice bath was removed and let the solution be stirred for 1 hour at 23° C. The reaction was monitored by TLC (1:3, EtOAc : Hexane). The resulting mixture was then separated, and the aqueous layer extracted with DCM. The combined organic layers were washed with saturated NaHCO3 and brine, dried over anhydrous Na₂SO₄, and evaporated. The crude product was purified by flash column chromatography with EtOAc : Hexane (1:5) as eluents to obtain the product I4 (65 mg, 0.21 mmol, 57 %, over two steps) as white solid.

I4. ¹H NMR (400 MHz, CD2Cl2) δ 6.98 (s, 2 H), 6.70 (s, 2 H), 3.83 (s, 3 H), 2.34 (s, 9 H), 2.28 (s, 6 H). ¹³C NMR (100 MHz, CD₂Cl₂) δ 152.16, 151.95, 150.35, 150.13 (The highest four peaks).

The obtained I4 (65 mg, 0.21 mmol) were dissolved in 2 ml dry DCM in a flask. The flask was then cooled to 0° C. To the stirring suspension, 1 M BBr3 in DCM (0.5 ml, 0.5 mmol) was added dropwise. The stirred solution was kept at 0° C. for 1 hour, then allowed to warm to 23° C. and reacted overnight. After reaction, at 0° C. the suspension was quenched slowly with saturated NH₄Cl solution, extracted with DCM, washed with brine and dried over anhydrous Na₂SO₄. The obtained product I5 was used without further purification.

The product I5, K₂CO₃ (56 mg, 0.4 mmol) and (3-Bromopropyl)triphenylphosphonium bromide (279 mg, 0.6 mmol) were stirred in 5 ml DMF at 70° C. for 1 hour. Then the reaction solution was filtered and quenched with saturated NH₄Cl solution. The solution was extracted by EtOAc and concentrated. The crude product was subjected to column chromatography with (MeOH + AcOH) in DCM (10% + 2.5%) as eluents to obtain Me-Mito¹³C (16, 75 mg, 0.11 mmol, 52% over two steps) as pale white solid.

¹H NMR (400 MHz, CD₂Cl₂) δ 7.90 - 7.76 (m, 9 H), 7.75 - 7.65 (m, 6 H), 6.96 (s, 2 H), 6.69 (s, 2 H), 4.32 (t, J = 5.9 Hz, 2 H), 4.10 - 3.84 (m, 2 H), 2.38 - 2.22 (m, 15 H), 2.22 - 2.14 (m, 2 H).

¹³ C NMR (100 MHz, CD₂Cl₂) δ 161.25, 151.98, 151.76, 150.57, 150.35, 142.76, 142.00, 139.36, 135.64, 134.33, 134.23, 131.01, 130.88, 129.08, 119.17, 118.31, 114.47, 67.32, 67.15, 23.29, 21.64, 21.22, 20.82, 20.36, 19.84.

HRMS (ESI, m/z): calcd. for [C₃₉¹³C₂H₄₀O₂P]⁺ (M⁺), 597.2828; found, 597.2838.

### Example 2 Imaging Experimental Procedures

This Example described general imaging experimental procedures and characterization methods used in the embodiments described in the present disclosure.

### Stimulated Raman Scattering and Fluorescence Microscopy

An integrated laser (picoEMERALD, Applied Physics and Electronics, Inc.) was used as a light source for both pump and Stokes beams. It produces 2 ps pump (tunable from 770 nm - 990 nm, bandwidth 0.5 nm, spectral bandwidth ~ 7 cm-1 ) and Stokes (1031.2 nm, spectral bandwidth 10 cm-1 ) beams with 80 MHz repetition rate. Stokes beam is modulated at 20 MHz by an internal electro-optic modulator. The spatially and temporally overlapped pump and Stokes beams are introduced into an inverted multiphoton laser scanning microscopy (FV3000, Olympus), and then focused onto the sample by a 25X water objective (XLPLN25XWMP, 1.05 N.A., Olympus). Transmitted pump and Stokes beams are collected by a high N.A. condenser lens (oil immersion, 1.4 N.A., Olympus) and pass through a bandpass filter (893/209 BrightLine, 25 mm, AVR Optics) to filter out Stokes beam. A large area (10×10 mm) Si photodiode (S3590-09, Hamamatsu) is used to measure the remaining pump beam intensity. 64 V DC voltage was used on the photodiode to increase saturation threshold and reduce response time. The output current is terminated by a 50 Ω terminator and pre-filtered by an 19.2-23.6-MHz band-pass filter (BBP-21.4+, Mini-Circuits) to reduce laser and scanning noise. The signal is then demodulated by a lock-in amplifier (SR844, Stanford Research Systems) at the modulation frequency. The in-phase X output is fed back to the Olympus IO interface box (FV30-ANALOG) of the microscope. Image acquisition speed is limited by 30 µs time constant set for the lock-in amplifier. Correspondingly, we use 80 µs pixel dwell time, which gives a speed of 8.5 s frame-1 for a 320-by-320-pixel field of view. Laser powers are monitored through image acquisition by an internal power meter and power fluctuation are controlled within 5% by the laser system. 16-bit grey scale images are acquired by Fluoview software. SRS spectra were acquired by fixing the Stokes beam at 1031.2 nm and scanning the pump beam through the designated wavelength range point by point. 10 mM EdU (H2O) sample was used as a standard to give RIE of different probes. Fluorescence images were collected using the same Olympus FV3000 confocal microscope with CW laser excitation (405, 488, 561 and 640 nm, Coherent OBIS LX laser) and standard bandpass filter sets. The correlation coefficients (Pearson’s R value) between SRS images and fluorescence images were calculated using ‘Coloc 2’ tool of ImageJ.

### Spontaneous Raman Spectroscopy

To perform spontaneous Raman spectroscopy, spontaneous Raman spectra were acquired using an upright confocal Raman spectrometer (Horiba Raman microscope; Xplora plus). A 532 nm YAG laser is used to illuminate the sample with a power of 12 mW on sample through a 100 x, N.A. 0.9 objective (MPLANN; Olympus) with 100 µm slit and 500 µm hole. Spectro/Raman shift center was set to be 2000.04 cm-1 . With a 1200 grating, Raman shift ranges from 690.81 cm-1 to 3141.49 cm-1 was acquired to cover whole biological relevant Raman peaks. The acquired spectra were processed by the LabSpec 6 software for baseline correction.

### 365 Nm UV Irradiation

The 365 nm UV light used to activate cyclopropenones comes from a UV handlamp (Analytik Jena UVP EL Series Lamp, UVLS-24, 4 Watt, 2 UV 254/365 nm, typically used for TLC monitoring in organic synthesis). The power of 365 nm UV of this handlamp was measured to be 15 mW/cm2 on samples.

### UV-Vis Absorption Spectra

UV-Vis absorption spectra were recorded on a Varian Cary 50 UV-Vis Spectrophotometer (Agilent).

### (HP)LC-MS for Assessing the Stability of Cyclopropenone Probes in Physiological Condition

The assessed probe was first dissolved in DMSO and later diluted in 10 mM cysteine (Sigma, 168149, dissolved in PBS) solution or DMEM (Corning, 35-015-CV) medium. Analytical HPLC coupled with mass spectrometry (LC-MS) was performed on Agilent 1290 infinity LC system using ZORBAX RRHD Eclipse Plus C18, 95 Å, 2.1 x 50 mm, 1.8 µm column with Agilent 6140 Series Quadrupole LCMS / LC-MS / MSD / Mass Spectrometer System. The mobile phase is water (0.1% AcOH) and acetonitrile with running method of gradient 5%-95% acetonitrile (1 ml/min, 4 min for total running time). The mass spectrometry detection region ranges from 100 to 800 AMU. The data shown in the main figures and supplementary figures are the absorption (280.8 nm) intensity traces.

### Cell Culture

HeLa cells (ATCC) were cultured in DMEM (Corning, 10-013-CV), supplemented with 10% fetal bovine serum (Corning, 35-015-CV), and 1% penicillin-streptomycin (Sigma-Aldrich). Cultures were incubated in a water-saturated incubator at 37° C. with 5% CO₂. Cells were passaged every 3-5 days once confluence reached 80%. Cultured HeLa cells were seeded onto 14 mm glass-bottom microwell dishes (MatTek Corporation) and grew to 80-90% confluence before labeling.

### Live/dead Cell Viability Assay

This assay was performed using the LIVE/DEAD viability/cytotoxicity kit for mammalian cells (Molecular Probes L-3224). HeLa cell standards and HeLa cells with dye staining were incubated with 2 µM calcein AM and 4 µM ethidium homodimer-1 (EthD-1) working solution for 20 min at 37° C. before imaging.

### Live-Cell Photo-Uncaging Organelle Imaging

Mitochondria imaging: Cells were incubated in 16 µM Me-Mito (12) or Me-Mito¹³C (16) with complete DMEM medium for 40 minutes in the incubator. Cells were washed with PBS three times before imaging. For colocalization, 80 nM MitoTracker™ Deep Red (Invitrogen, M22426) was used to stain cells for 25 minutes. For photo-uncaging, 405 nm laser (OBIS LX 405 nm 50 mW Laser, the same 405 nm laser used below) was used to scan the selected field of view for 2 minutes (1.6 mW on samples).

Lysosomes imaging: Cells were incubated in 30 µM Me-Lyso (13) with complete DMEM medium for 2 hours in the incubator. Cells were washed with PBS three times before imaging. For colocalization, 0.4 x LysoView™ 488 (Biotium, #70067) was used to stain cells for 30 minutes. For photo-uncaging, 405 nm laser was used to scan the selected field of view for 2 minutes (1.6 mW).

ER imaging: Cells were incubated in 40 µM Me-ER (14) with complete DMEM medium for 2 hours in the incubator. Cells were washed with PBS three times before imaging. For colocalization, 0.5 µM ER-Tracker™ Red (Invitrogen, E34250) was used to stain cells for 1 hour. For photo-uncaging, 405 nm laser was used to scan the selected field of view for 2 minutes (1.6 mW).

Lipid droplets imaging by Me-LD (15): Cells were incubated in 20 µM Me-LD (15) with complete DMEM medium for 30 minutes in the incubator. Cells were washed with PBS three times before imaging. For colocalization, 0.5 x LipidSpot™ 610 (Biotium, #70069) was used to stain cells for 20 minutes. For photo-uncaging, 405 nm laser was used to scan the selected field of view for 2 minutes (1.6 mW).

Lipid droplets imaging by Cyclo-LD (17): Cells were washed with PBS for three times before labeling. Cells were then incubated in 20 µM Cyclo-LD (17) with PBS for 30 minutes in the incubator. Cells were washed with PBS three times before imaging. For colocalization, 0.5 x LipidSpot™ 610 (Biotium, #70069) was used to stain cells for 20 minutes. For photo-uncaging, 405 nm laser (1.6 mW) was used to scan the selected field of view for 2 minutes.

Two-color tracking with Me-LD (15) and Me-Mito¹³C (16): Cells were incubated in 10 µM Me-LD (15) and 20 µM Me-Mito13C (16) with complete DMEM medium for 30 minutes in the incubator. Cells were washed with PBS three times before imaging. For selective uncaging, 405 nm laser was used to scan the selected field of view for 15 seconds (0.8 mW).

Three-color targeted photo-uncaging imaging with Cyclo-LD (17), Me-Lyso (13) and MeMito¹³C (16:. Cells were incubated in 40 µM Me-Lyso (13) with complete DMEM medium for 4 hours and 20 µM Me-Mito¹³C (16) with complete DMEM medium for 30 minutes in the incubator. Cells were washed with PBS three times. Cells were then incubated in 15 µM Cyclo-LD (17) with PBS for 15 minutes at room temperature. Cells were washed with PBS three times before imaging. For photo-uncaging, 405 nm laser was used to scan the selected field of view for 2 minutes (1.6 mW).

### Quantification of SRS Laser Uncaging.

Cells were incubated in 20 µM Me-Mito (12) with complete DMEM medium for 30 minutes in the incubator. Cells were washed with PBS three times before imaging. After taking one image at CH3 channel (2940 cm-1 ) at low powers (10 mW OPO and 10 mW Stokes, 80 µs/pixel), 10 frames of images at 2205 cm-1 were taken at various OPO and Stokes powers sequentially, followed by 405 nm illumination. The image acquired after 405 nm irradiation was assigned as 100% signal frame. The signal of each frame in the 10 images set was divided by the signal of 405 nm irradiated frame (100% signal frame) to obtain its percentage. Our criterion for the powers used were to let the intensity gain of each frame be less than 1.5%, so we chose 25 mW OPO and 60 mW Stokes for imaging.

### Example 3 Synthesis and Spectroscopic Characterizations of Model Cyclopropenone-caging Systems

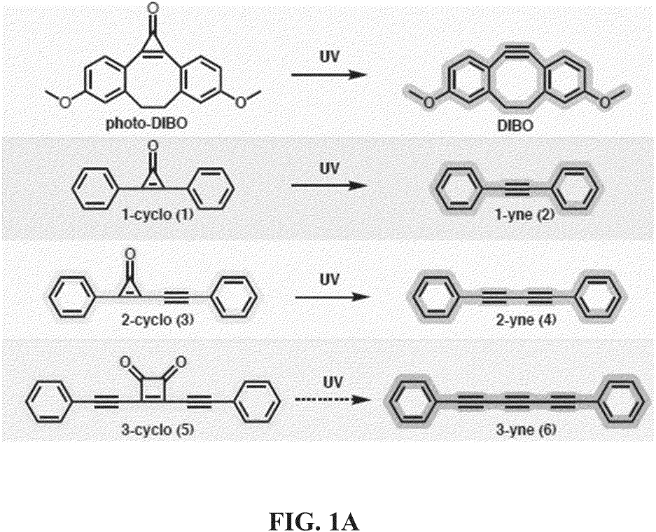

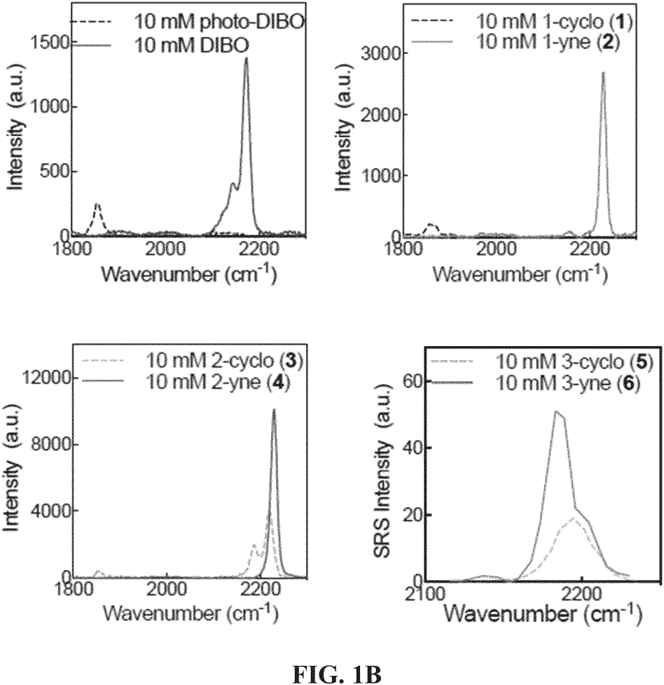

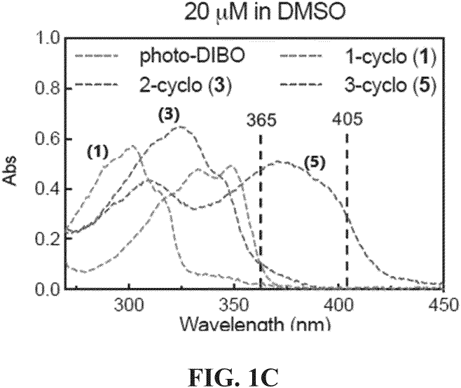

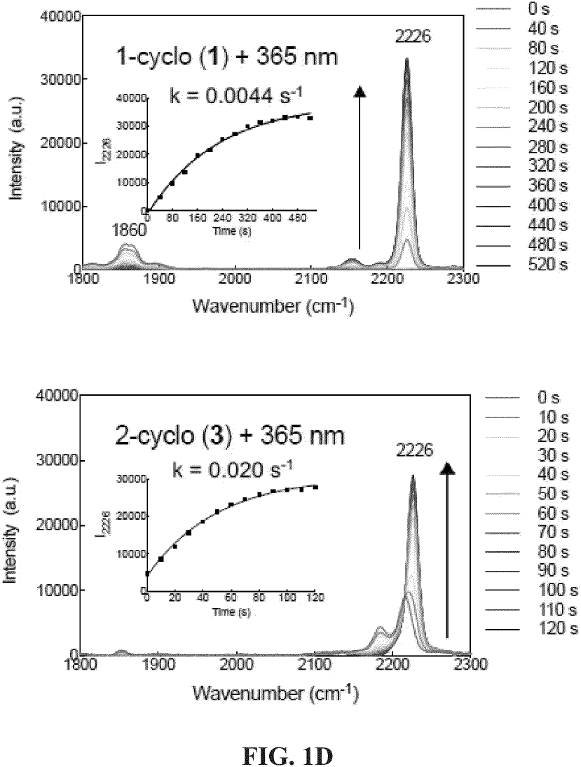

Cyclopropenone caging has been previously adopted for photoactivation and subsequent biorthogonal labeling of fluorophores in fluorescence microscopy. For examples, copperfree click labeling of dibenzocyclooctynes (DIBO) released from photoactivated diarylcyclopropenones (photo-DIBO) has been demonstrated for surface and cellular labeling and imaging. Herein the alkyne Raman signal generation from photo-DIBO light-activation was first measured (FIG. 1A). In the cell silent region, photo-DIBO only has a weak Raman peak around 1851 cm⁻¹ while photochemically generated DIBO by UV shows a characteristic alkyne peak at 2171 cm⁻¹ with significantly enhanced Raman intensity (FIG. 1B, dashed black vs solid green spectra). Compared to 5-ethynyl-2′-deoxyuridine (EdU), the well-adopted benchmark for Raman intensity quantification, the relative Raman intensity to EdU (RIE) of the strained alkyne DIBO is only 1.8 (Table 1). Compared with DIBO, the planar diphenylacetylene (1-yne, 2, FIG. 1A) exhibits larger alkyne Raman signals. Given this, it is reasoned that diphenylcyclopropenone (i.e. cyclopropenone-caged 1-yne, 1-cyclo, 1, FIG. 1A) should be a better photoactivatable Raman scaffold with higher Raman sensitivity after uncaging. Indeed, UV-uncaging of 1-cyclo (1), which started from a similarly weak Raman peak at 1860 cm⁻¹ from photo-DIBO, produced an intense sharp peak for 1-yne (2) at 2226 cm⁻¹ (FIG. 1B, dashed black vs solid blue spectra) with an improved RIE values to about 4

TABLE 1

Photophysics characterization of molecules in model systems

| | | | | | | | | | | |
|---|---|---|---|---|---|---|---|---|---|---|
| Species | Photo-DIBO | DIBO | 1-cyclo (1) | 1-yne (2) | 2-cyclo (3) | 2-yne (4) | 3-cyclo (5) | 3-yne (6) | 9 | 10 |
| Raman Peak (cm⁻ ¹) | 1851 | 2171 | 1860 | 2226 | 2218 | 2226 | 2194 | 2185 | 1850 | 2205 |
| RIE | 0.34 | 1.8 | 0.37 | 4.0 | 7.5 | 19 | 13 | 31 | 0.89 | 7.7 |
| Abs (nm) | 333 | 323 | 302 | 283 | 324 | 310 | 371 | 337 | 339 | 323 |
| ε_{abs} (M⁻¹cm⁻¹) | 23900 | 24200 | 28600 | 32300 | 32400 | 35700 | 25500 | 28100 | 13300 | 3680 0 |

Polyynes are an established palette of highly sensitive and multiplexable Raman probes. Next, the feasibility of constructing polyynes-based cyclopropenones to obtain even higher after-conversion Raman intensity was explored. Cyclopropenone-caged 2-yne (i.e. 2-cyclo, 3, FIG. 1A) was synthesized and it was found that it also possesses superb UV-uncaging photochemistry similar to that of 1-cyclo (1). Photoconversion from 2-cyclo (3) to 2-yne (4) indeed generated a sharp alkyne peak with an RIE of 19 (Table 1). However, the spectra of 2-cyclo (3) and 2-yne (4) present a partial overlap (FIG. 1B, dashed vs solid purple spectra), which is less desirable for backgroundfree detection. Attempt was further made to synthesize 3-yne-based cyclopropenone, but the product was highly unstable at room temperature and decomposed quickly during concentrating. As an alternative, thermally stable 3,4-Bis(phenylethynyl)-3-cyclobutene-1,2-dione was chosen as the target molecule for caged 3-yne (i.e. 3-cyclo, 5, FIG. 1A), which was reported to yield 3-yne (6) by pyrolytic loss of two carbonyl groups, and was suggested to also undergo photolysis process to give the same product. However, no activated Raman signals from synthesized 3-cyclo (5) were observed observe after illuminations from a handheld UV-lamp (254 nm and 365 nm) or 405 nm continuouswave (CW) laser. Additionally, characterizations from synthesized pure 3-cyclo (5) and 3-yne (6) showed that they also present an undesirable partial spectral overlap (FIG. 1B, dashed vs solid red spectra, the SRS spectra are shown instead of spontaneous Raman spectra due to the large autofluorescence background of 3-cyclo, 5).

Based on the above spectroscopy characterizations on photo-DIBO and 1- to 3-cyclo (1, 3, 5) model compounds, 1-cyclo (1) and 2-cyclo (3) present as better candidates judging from photo-conversion capabilities and uncaged alkyne intensity (Table 1). The sizes of 1-cyclo (1) and 2-cyclo (3) are also small, thus would only exhibit minor labeling perturbation to biological targets. Moreover, the uncaged 1-yne (2) and 2-yne (4) are immune to photobleaching, making quantification more straightforward and reliable.

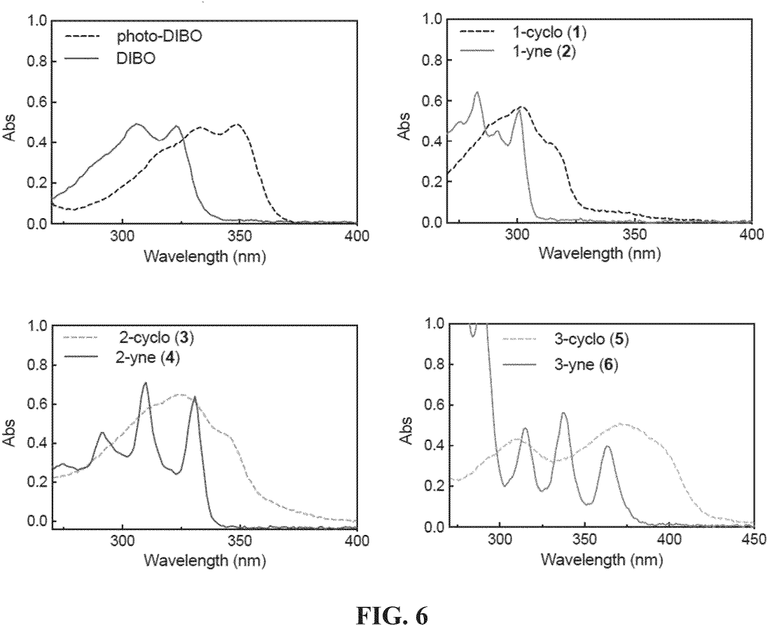

Next, the photo-uncaging kinetics for both 1-cyclo (1) and 2-cyclo (3) were compared. The absorption spectra of all four caged model compounds are shown in FIG. 1C. Although both 365 nm and 405 nm excitations only fall around the tails of the spectra, either handheld 365 nm UV-lamp or 405 nm CW laser illumination can effectively uncage both 1-cyclo (1) and 2-cyclo (3). With 365 nm lamp illumination, the characteristic peaks of cyclopropenones (~1860 cm⁻¹) continue to drop while the alkyne peaks (2226 cm⁻¹) gradually increase for both molecules (FIG. 1D). 2-cyclo (3, k= 0.020 s⁻¹) showed uncaging kinetics about 5-fold faster than that of 1-cyclo (1, k= 0.0044 s⁻¹, with a reported quantum yield of 0.78), consistent with its higher molar extinction coefficient at 365 nm (FIG. 1C). The progression of the UV-activation reactions could also be confirmed by the change in the corresponding absorption spectra (FIG. 6).

### Example 4 Probe Engineering for Improved Chemical Stability In The Cellular Environment and Enhanced Raman Sensitivity

This example describes the process of engineering probes for live-cell photoactivatable Raman imaging based on the model cyclopropenone-caging system described in Example 3.

Although 2-cyclo (3) has higher uncaged Raman signals and faster uncaging kinetics than 1-cyclo (1), attempts to functionalize 2-cyclo (3) were not successful due to the instability of 2-cyclo core under the reaction conditions. Additionally, the background Raman signal from 2-cyclo (3) before photoactivation also complicates imaging analysis (FIG. 1B, dashed vs solid purple spectra). Hence, this example derivatizes 1-cyclo (1) as functional imaging probes in live cells.

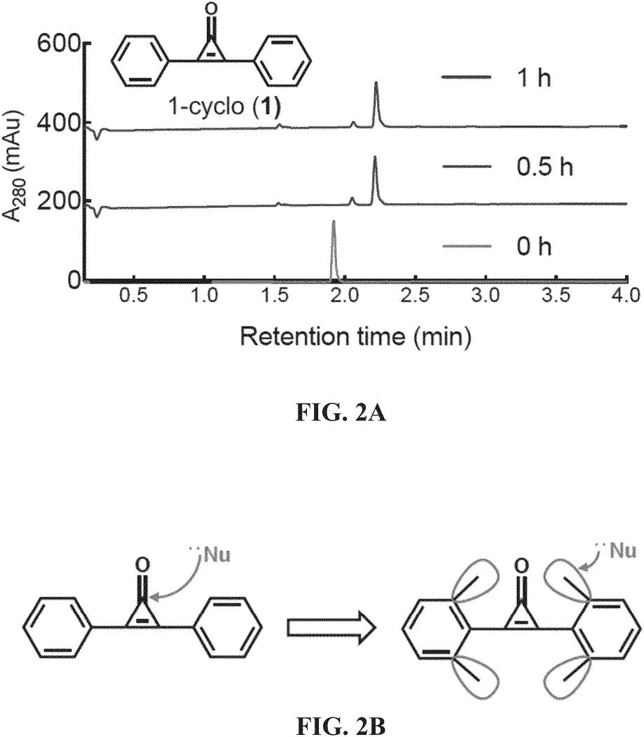

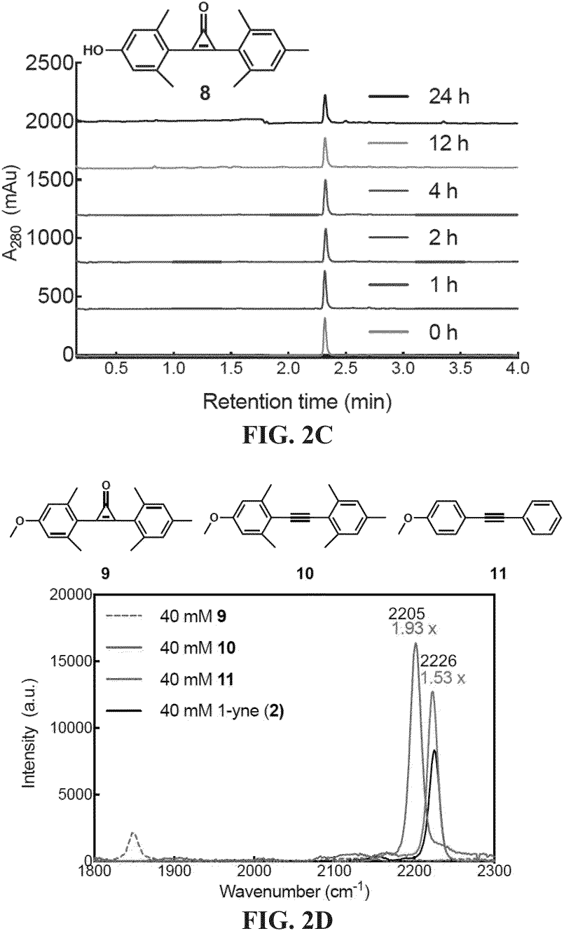

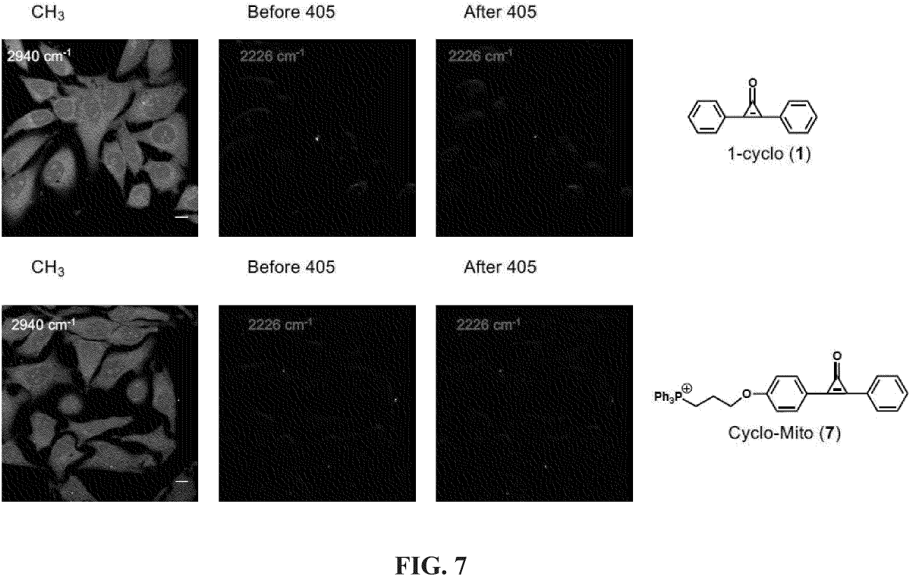

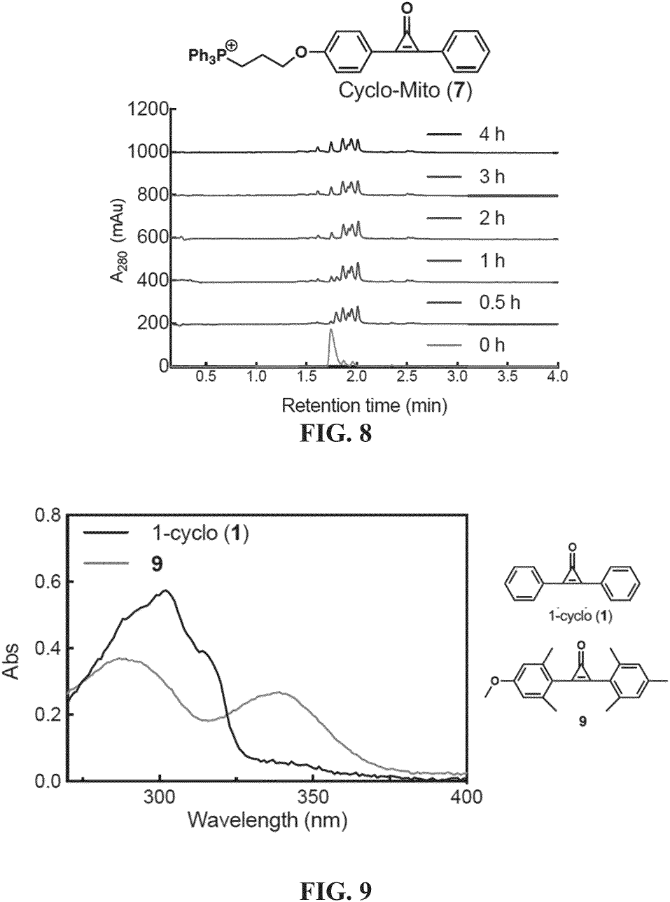

Nonetheless, it was found that 1-cyclo (1) significantly suffers from instability in the cellular environment. Either 1-cyclo (1) or functionalized 1-cyclo with a mitochondria targeting group (i.e. Cyclo-Mito, 7) did not produce any observable uncaging signals in labeled live cells (FIG. 7). It was hypothesized that this was likely because the cyclopropenone structures are susceptible to nucleophilic attack, leading to ring-opened α,β-unsaturated acids derivatives. Cysteines, glutathione, and other biological thiols are strong nucleophiles and could easily deactivate cyclopropenones. The stability of 1-cyclo (1) in 10 mM cysteine phosphate-buffered saline (PBS) at 37° C. was therefore tested, mimicking physiological conditions. HPLC analysis showed that both 1-cyclo (1) and Cyclo-Mito (7) completely decomposed within 30 minutes (FIG. 2A and FIG. 8). The coupled mass spectrometry indicated that the byproduct was cysteine adduct, consistent with previous report.

To test if incorporating alkyl groups around cyclopropenones shield the strained ketones from nucleophilic attack and improve the stability of 1-cyclo (1) inside cells (FIG. 2B), methyl-shielded 1-cyclo with Friedel-Crafts alkylation of aromatic compounds (Scheme 1) was synthesized. The phenol alcohol handle was introduced to link cellular targeting groups. HPLC trace confirmed that the methylated 1-cyclo (i.e. 8, Scheme 1) presented substantially improved stability to cysteine attack even for incubation up to 24 hours (FIG. 2C). It was further found that methylated cyclopropenones not only have improved stability to nucleophiles, but also have enhanced uncaging Raman signals. FIG. 2D shows that methylation and phenolation of the aromatic rings introduced an intensity increase (1.93 times) and a slight peak red-shift (21 cm⁻¹) to 2205 cm⁻¹ for the activated alkyne peak compared with the unfunctionalized 1-cyclo (the solid black spectrum from 1-yne vs the red solid spectrum from 10). In addition to the Raman spectral shift, the absorption spectrum of 9 also undergoes a red-shift to close to that from 2-cyclo (3), implying improved UV photoactivation kinetics (FIG. 9).

Scheme 1. Synthesis of methyl-shielded 1-cyclo (8) and the corresponding organelle targeting probes (e.g. the mitochondria targeting probe, Me-Mito, 12)

### Example 5 Organelle-Targeted Probe Engineering for Live-cell Photoactivatable Raman Imaging

This example describes the process of engineering and synthesizing organelle-targeted cyclopropenone probes and applications in live-cell imaging.

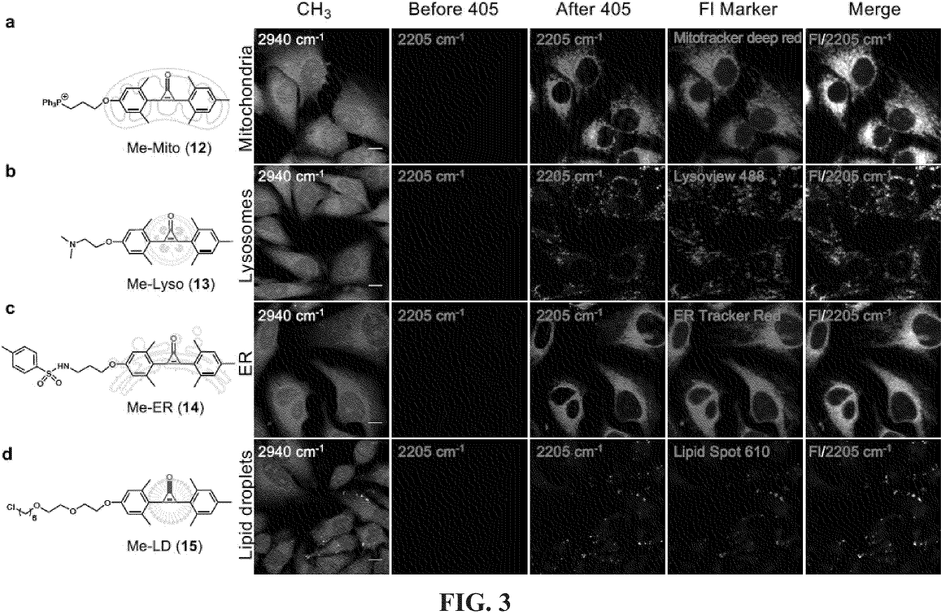

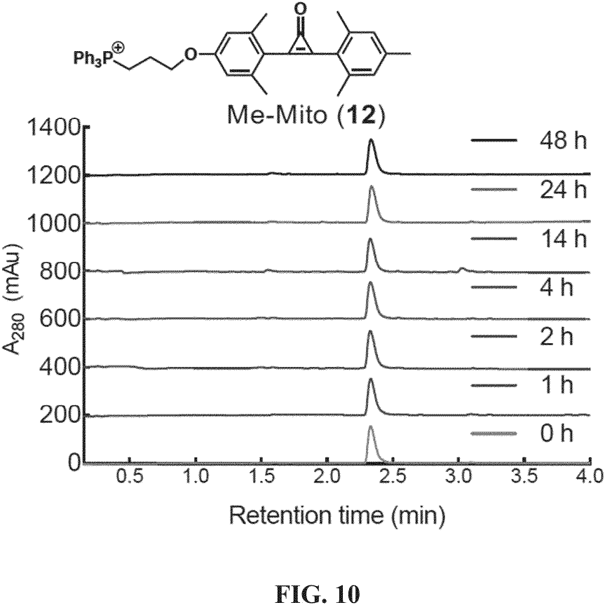

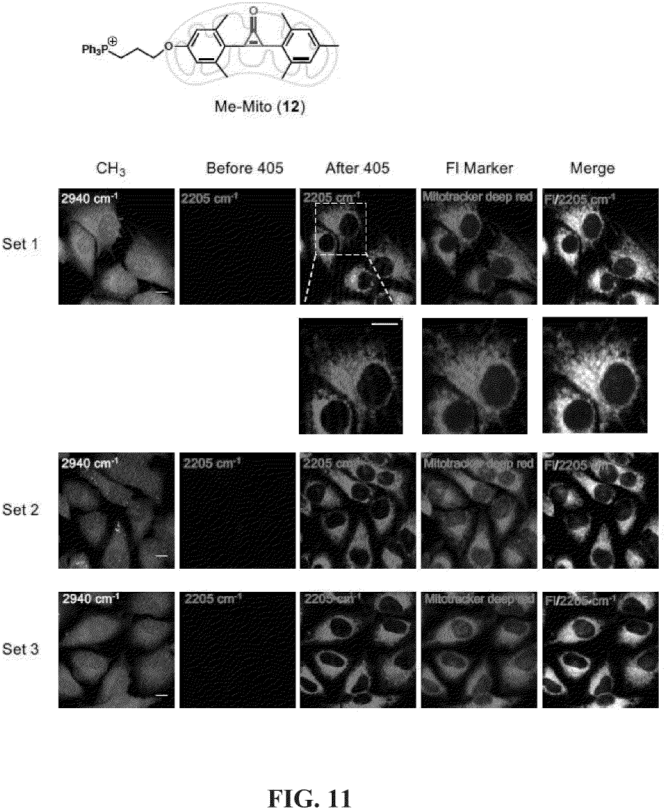

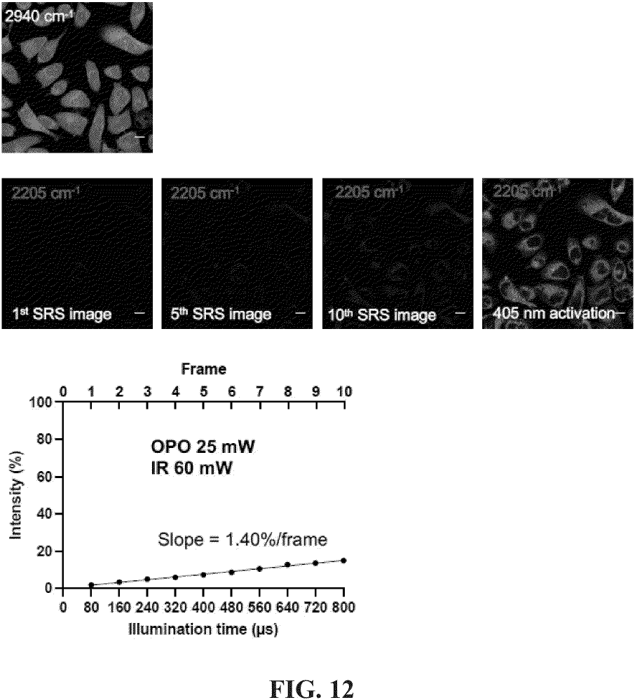

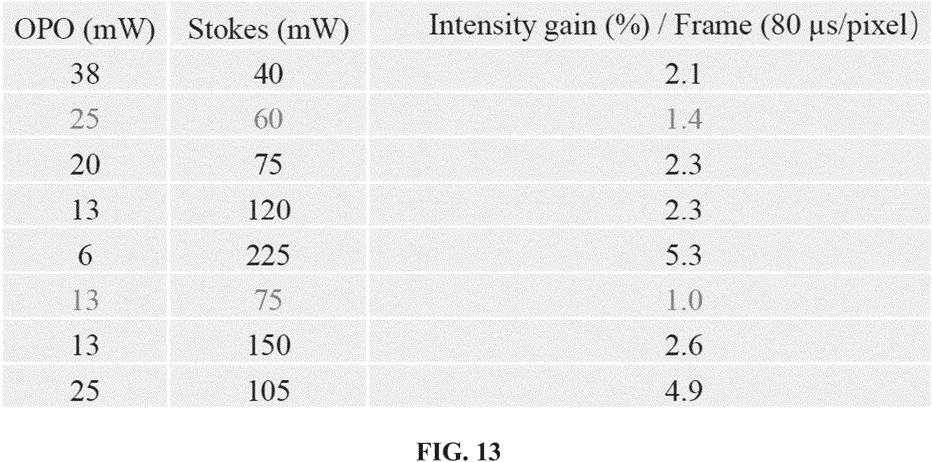

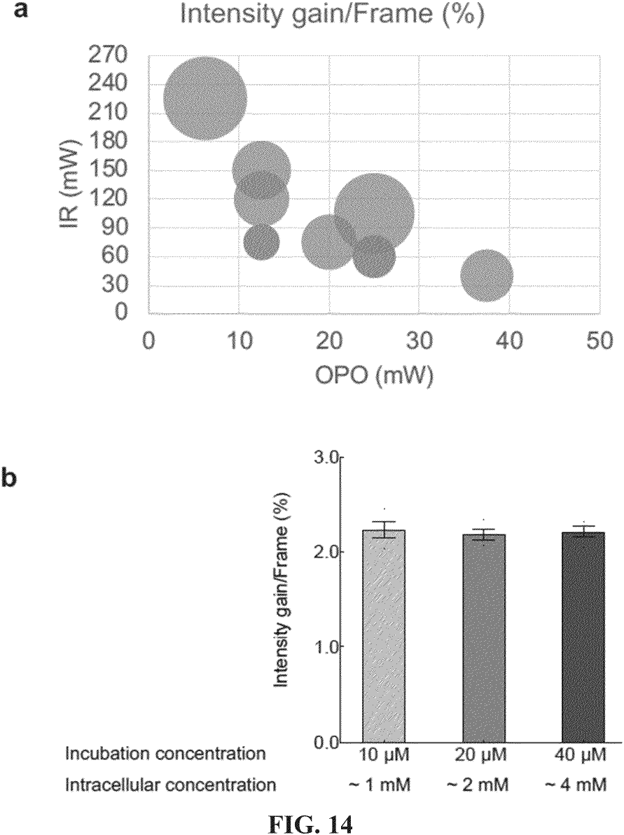

After the methylated phenyl capped 1-cyclo was established as a suitable uncaging scaffold for intracellular investigations, organelle-targeted cyclopropenone probes were then synthesized and applied to live-cell imaging. The Me-Mito (12) probe uses positively charged triphenylphosphonium (TPP⁺) as a mitochondria targeting group (Scheme 1 & FIG. 3, panel a). It was first confirmed via HPLC that the synthesized Me-Mito (12) was stable in 10 mM cysteine solution for over 48 hours (FIG. 10). Next, cells were co-incubated with Me-Mito (12) and a commercial mitochondria fluorescence marker (Mitotracker deep red) and live-cell imaging was performed. It was validated that the caged Me-Mito (12) presented a clean background in cells before UV-activation (FIG. 3, panel a, Before 405, 2205 cm⁻¹; the CH₃ SRS image at 2940 cm⁻¹ outlines the cell morphology for the same set of cells). After two minutes of 405 nm laser illumination, a bright mitochondria pattern was observed by SRS imaging at the alkyne channel (FIG. 3, panel a, After 405, 2205 cm⁻¹), implying a high signal-to-background ratio. The labeling specificity by Me-Mito (12) was further verified by the co-localization with the fluorescence maker (FIG. 3, panel a, Fl marker and Merge; the magnified images, additional sets of activation and correlation images, and Pearson’s R values are shown in FIG. 11). It is worth pointing out that, it was found that high-power SRS lasers could very slowly activate the Me-Mito (12, FIG. 12), likely due to multi-photon activation from the pump (~ 800 nm, 2 ps) or Stokes (1031 nm, 2 ps) laser alone and also from their combined excitation based on a series of activation-power relationship characterizations (FIG. 13 and FIG. 14, panel a). It was confirmed that the activation by SRS lasers is independent of probe concentration (FIG. 14, panel b) and selected 25 mW and 60 mW as suitable SRS imaging powers for the pump and Stokes beam respectively (FIG. 14, panel a), which is high enough to maintain a decent imaging quality, but low enough to both yield a low side photoactivation rate by SRS lasers (close to one percent per frame with 80 µs pixel dwell time) and maintain minimum photo-toxicity. It was also envisioned that the photoactivation rate from SRS lasers would be much-lowered with a faster scanning rate (i.e. lower pixel dwell time).

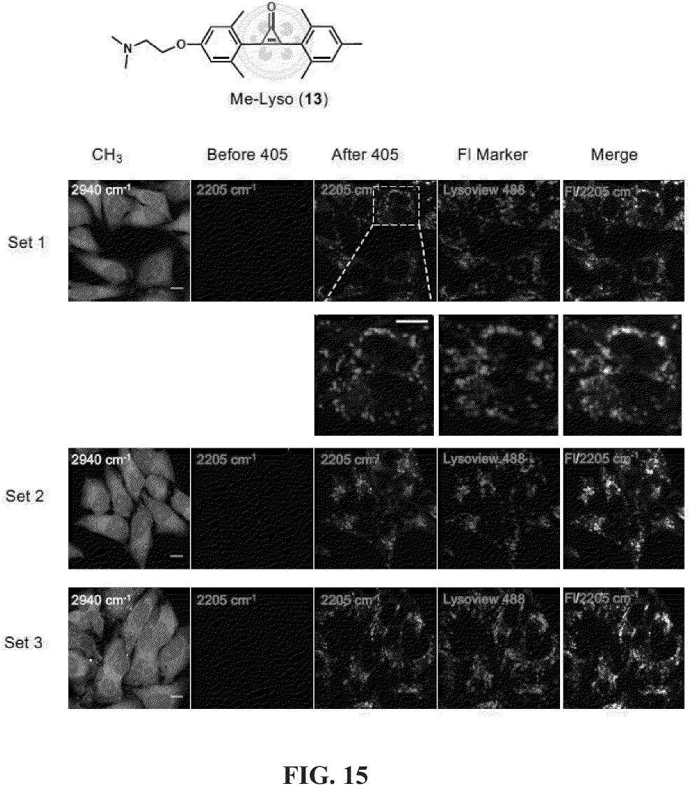

Similarly, the lysosome imaging probe (Me-Lyso, 13, FIG. 3, panel b) was designed by introducing a basic dimethylamine group for acidic organelle targeting through similar chemistry as shown in Scheme 1 (detailed reaction conditions listed in Example 1). The clean background (FIG. 3, panel b, Before 405, 2205 cm⁻¹), the achievable high photoactivation ratio (FIG. 3, panel b, After 405, 2205 cm⁻¹), and desired co-localization (FIG. 3, panel b, Merge) for proper lysosomes targeting from Me-Lyso (13) and the commercial Lysoview 488 fluorophore co-labeled live cells (FIG. 3, panel b and FIG. 15) were also confirmed.

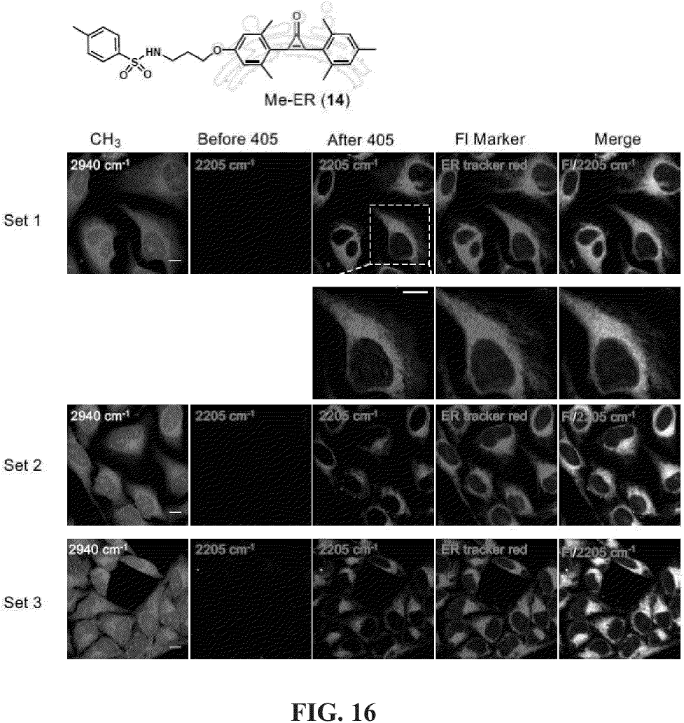

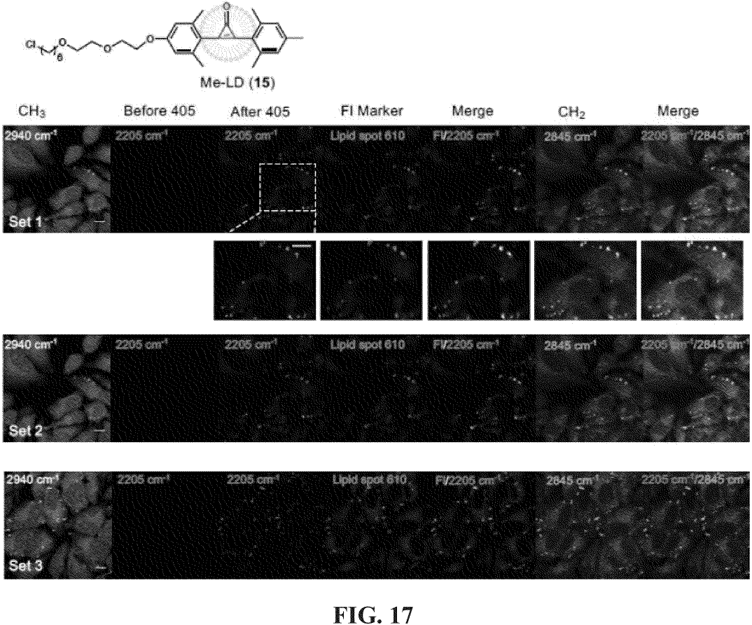

Furthermore, the endoplasmic reticulum imaging probe (Me-ER, 14, FIG. 3, panel c) was designed by adding the methyl sulphonamide group and highly specific targeting, photoactivation and imaging performances (FIG. 3, panel c & FIG. 16) were confirmed. Unexpectedly, when setting out to design a HaloTag ligand carrying cyclopropenone probe for protein imaging, it was instead found that this probe showed high-specificity for staining lipid droplets with preserved photoactivation properties. Interestingly, it quickly stained lipids droplets within 15 minutes, much faster than previously reported carboxylate-terminated C12 alkyl polyyne Raman probe, which requires overnight labeling. The probe was hence named Me-LD (15, FIG. 3, panel d) after confirming its specificity in reference to fluorescence co-localization of lipid droplets marker Lipid Spot 610 (FIG. 3, panel d and FIG. 17).

The above demonstrations showed that the methylated cyclopropenone is a well suitable motif for general design of live-cell compatible photo-uncaging Raman probes.

### Example 6 Live-cell Photoactivatable Multiplex Imaging

This example describes the development of photoactivatable organelle-targeting Raman probes for photoactivatable multiplex imaging.

The isotope editing strategy was adapted for Raman color shift. With bis-¹³C isotope substitution on the Me-Mito (12) probe, the Me-Mito-¹³C (16) showed a drastic peak shift from the original 2205 cm⁻¹ to 2125 cm⁻¹ after uncaging (FIGS. 4A-B, blue color-coded). Therefore Me-Mito-¹³C (16) provided a well-resolvable color in addition to that offered by the non-isotopeedited probe such as Me-Lyso (13, FIGS. 4A-B, magenta color-coded). It is fully expected that mono¹³C substitution would likely offer additional resolvable colors within the 80 cm⁻¹ spectral gap similar to previously reported. Nevertheless, the synthetic challenges and costs can also be higher.

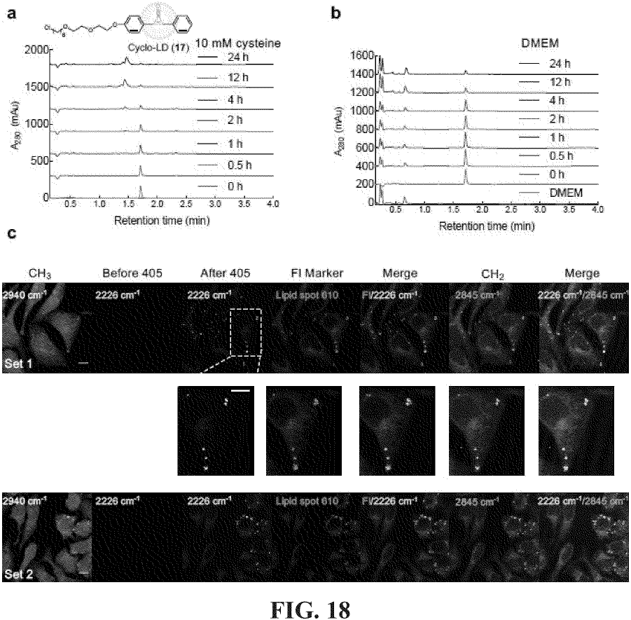

To generate more photoactivatable Raman colors, the utility of the 21 cm⁻¹ peak shift from unmethylated cyclopropenone (FIGS. 2D, 11 vs 10) was explored next. It was revealed that the unmethylated cyclopropenone, once functionalized as the hydrophobic Cyclo-LD probe (17, FIG. 4, panel a, green color-coded), showed a much-increased stability to thiol-containing buffers (FIG. 18, panels a-b) and preserved the fast lipid droplets labeling property. Its suitability for live-cell lipid droplet imaging is also confirmed by fluorescence co-staining (FIG. 18, panel c). Together with the un-edited Me-Lyso (13) and the Me-Mito-¹³C (16), Cyclo-LD (17) could be adopted as the third photoactivatable color for intracellular multiplex imaging with minimal spectral cross-talk (FIG. 4, panels b-c).

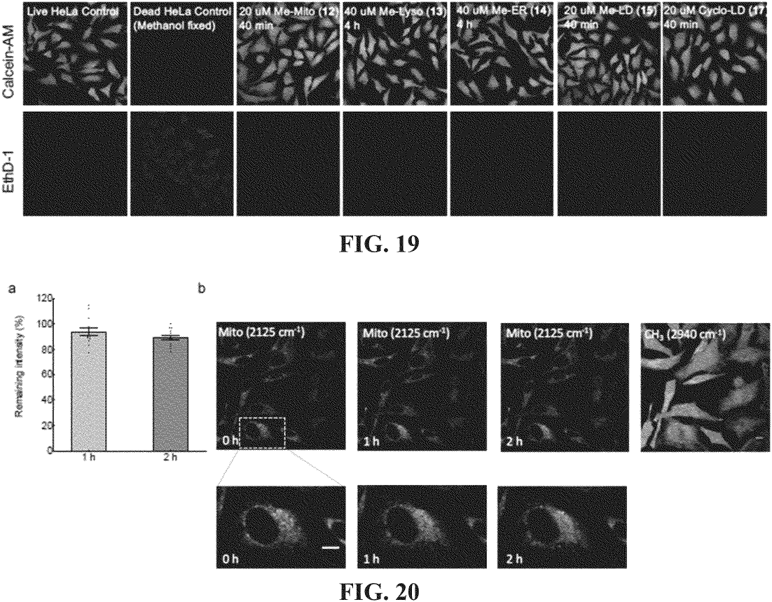

After confirming the minimal cytotoxicity of all cyclopropenone imaging probes (FIG. 19), three-color co-labeling for mitochondria (Me-Mito-¹³C, 16), lysosomes (Me-Lyso, 13) and lipid droplets (Cyclo-LD, 17) on the same set of live cells (FIG. 4, panel d) were further demonstrated. The photoactivatable multiplex imaging of these intracellular targets presents clear contrast from each individual channel without the need of linear unmixing (FIG. 4, panel d). The well-maintained cellular morphology throughout photoactivation and multiplex imaging also proved the superior live-cell compatibility of this strategy.

### Example 7 Intracellular Photoactivation and Multiplex Tracking

This example describes the application of photoactivatable Raman probes in illuminating subcellular and single-cell dynamics.

Taking advantage of the high spatial-temporal control feature, tracking subcellular structures and single cells could realize the full potential of photoactivatable imaging. In addition, multiplex activation of single cells would illuminate complex cell-to-cell interactions and facilitate cell profiling. The non-invasive multiplex SRS imaging with photoactivatable probes would be highly appealing for such applications. Here, this example demonstrates that this approach could illuminate subcellular and single-cell dynamics with pulse-chase photoactivation and imaging.

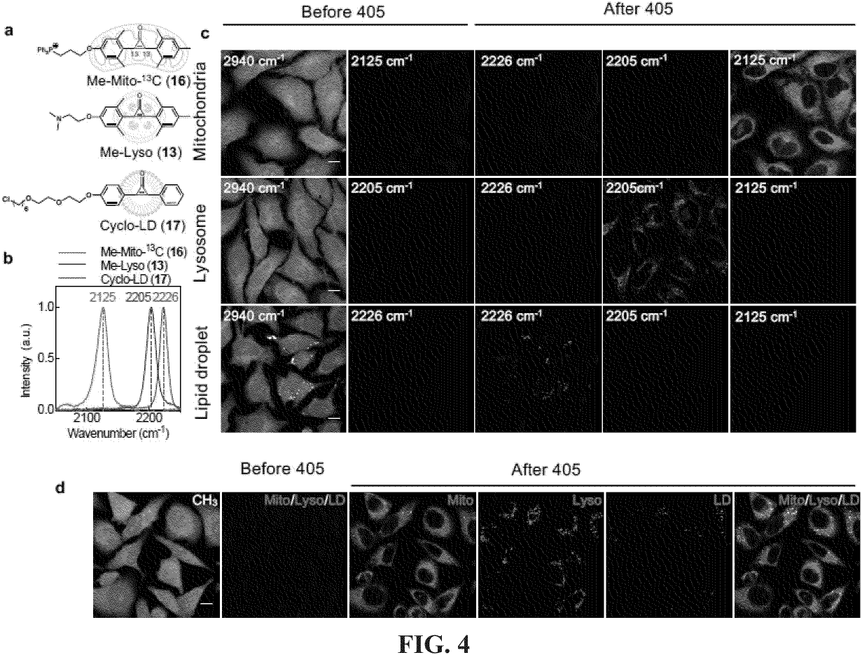

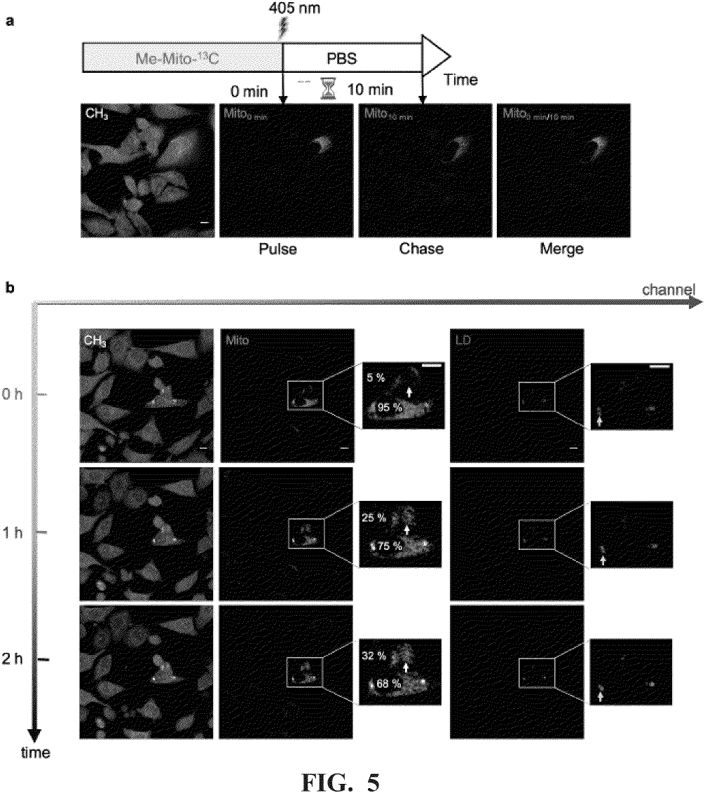

The high mitochondria-labeling specificity of the mitochondria probe was confirmed for relatively long-term pulse-chase tracking applications. More than 85% of activated Me-Mito-¹³C (16) signals retained in live HeLa cells after 2 hours of chasing (FIG. 10, panel a) with well-maintained mitochondria labeling pattern (FIG. 20, panel b). In live HeLa cells labeled with Me-Mito-¹³C (16), 405 nm laser was first used to pulse-activate the selected region of a single cell for 20 seconds (FIG. 5A, CH₃, the white dashed box indicates the activation area). Immediately after photoactivation, SRS imaging at 2125 cm⁻¹ well resolved the distribution of mitochondria only from this activated subcellular region (FIG. 5A, Mito₀ ₘᵢₙ, Pulse), implying high spatial selectivity. After only a 10-minute chase period, SRS imaging at the same channel (2125 cm⁻¹) captured fast redistribution of activated mitochondria signals (FIG. 5A, Mito₁₀ ₘᵢₙ, Chase). The merged image of these two time spots (FIG. 5A, Merge) clearly showed the quick diffusion of mitochondria, indicating dynamic mitochondria fission and fusion in live cells, consistent with the recent report.

In another set of live HeLa cells co-labeled with Me-Mito-¹³C (16) and Me-LD (15) for activation and multiplex tracking, similar pulse-chase experiments were carried out (FIG. 5B). After activating a single cell (FIG. 5B, CH₃, the outline of the cell was indicated by white dashed box), two-channel SRS imaging of mitochondria (FIG. 5B, Mito, at 2125 cm⁻¹) and lipid droplets (FIG. 5B, LD, at 2205 cm⁻¹) was immediately acquired (0 h). Subsequent chase imaging was performed at an interval of one hour for up to two hours (1 h, 2 h). In the mitochondria channel (FIG. 5B, Mito), it captured a likely material transfer process from the photoactivated cell to the neighboring cell (indicated by the white arrow in the magnified images, the relative signals of the neighboring cell increased from 5% to 32% after 2 hours). In the parallel droplet channel (FIG. 5B, LD), the chase images also demonstrated that the lipid droplets in the left side of the photoactivated cell (indicated by the yellow arrow in the magnified images) were approaching each other and were probably in the process of merging. For further improvement, we envision a covalently labeling probe (e.g. Me-Mito modified with a chloromethyl anchoring group) would allow unequivocal long-term tracking in more complicated biological systems. The multiplex tracking with high spatial-temporal control hence present promises to understand complex intra- and inter-cellular interactions.

### Example 8 Multiplex Imaging of Interactions between Organelles

This example describes an exemplary procedure using organelle targeting cyclopropenone probes to study the organelle interaction in live cells such as lysosome-lipid droplet contact during lipophage.

Previous reports show that inhibition of rapamycin complex 1 (mTORC1) will initiate autophagosome. Lipid droplets will be degraded via lipophagy upon fusion with lysosomes. In this way, the co-localization of lipid droplets and lysosomes during mTORC1 inhibition was imaged and tracked with the multicolor photoactivatable probes described herein.

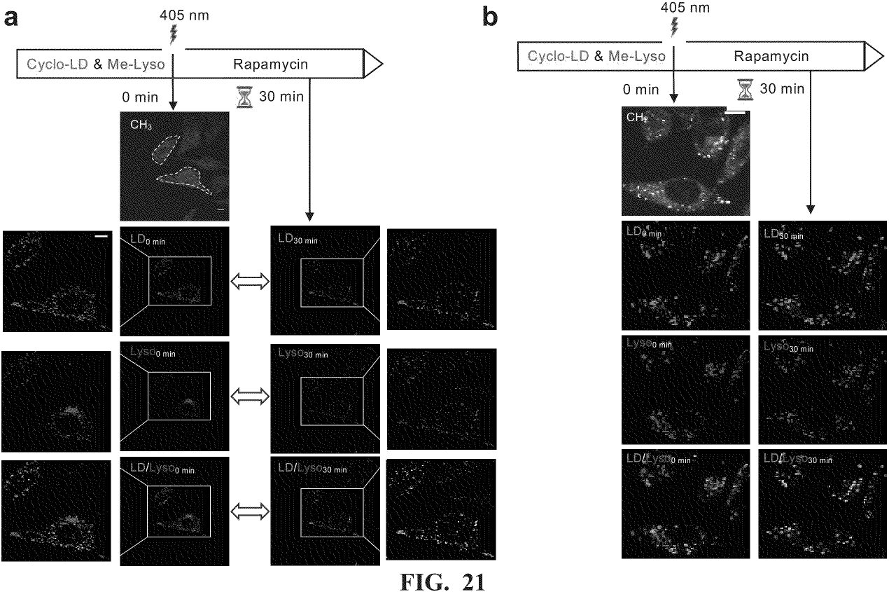

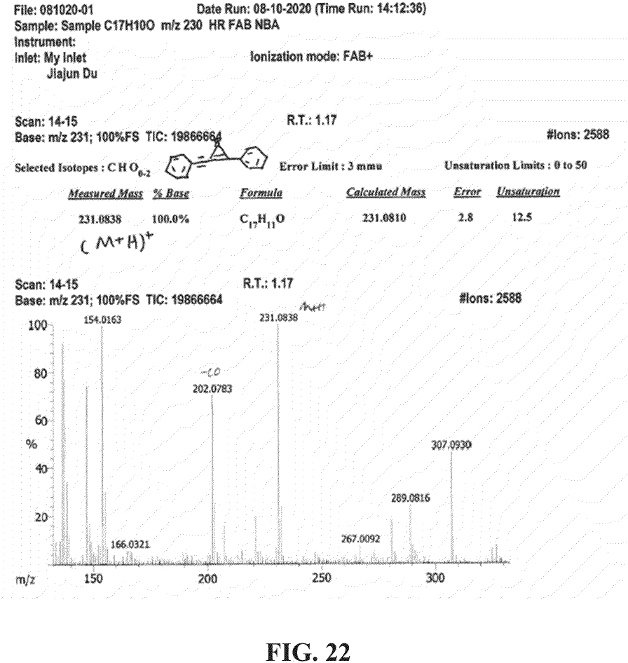

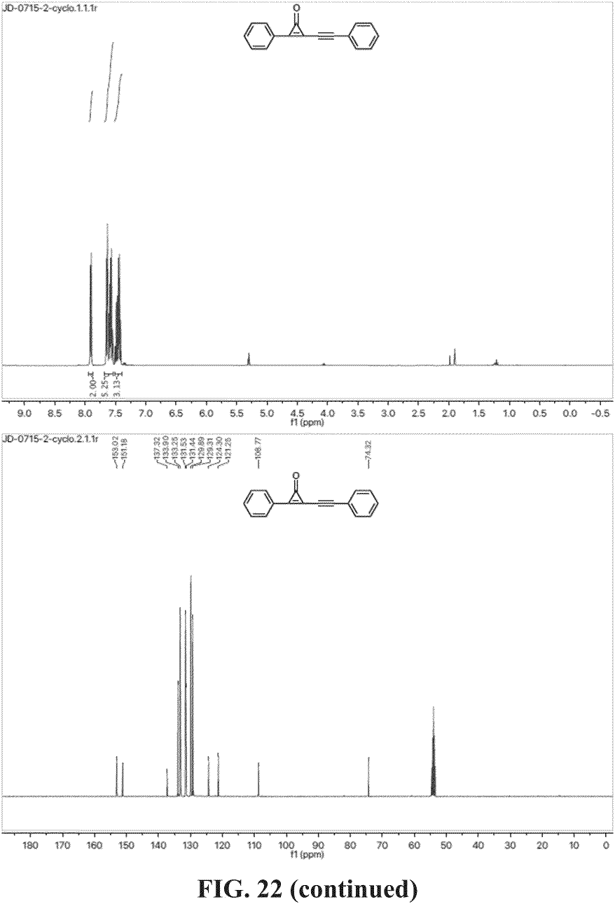

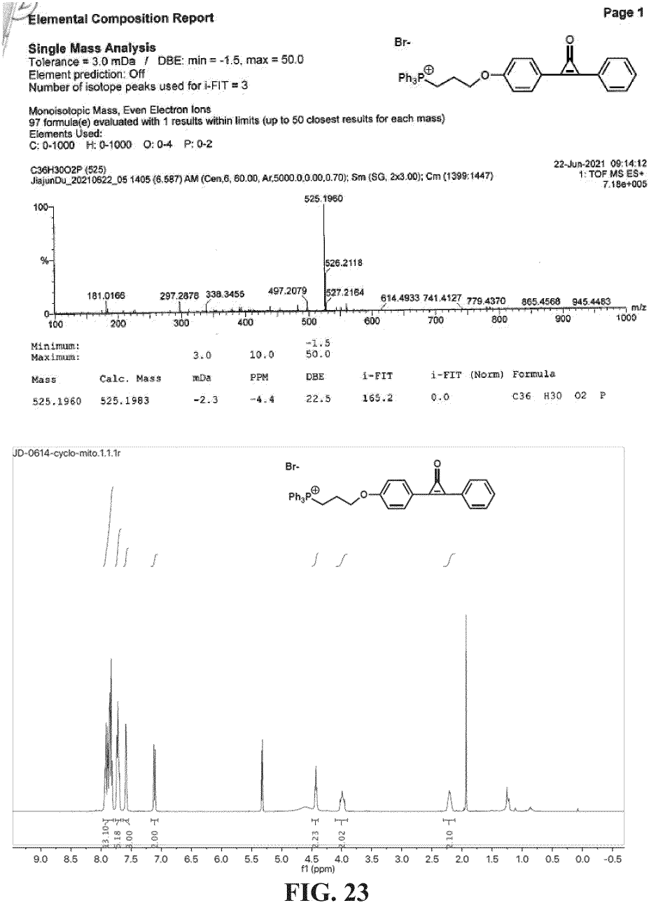

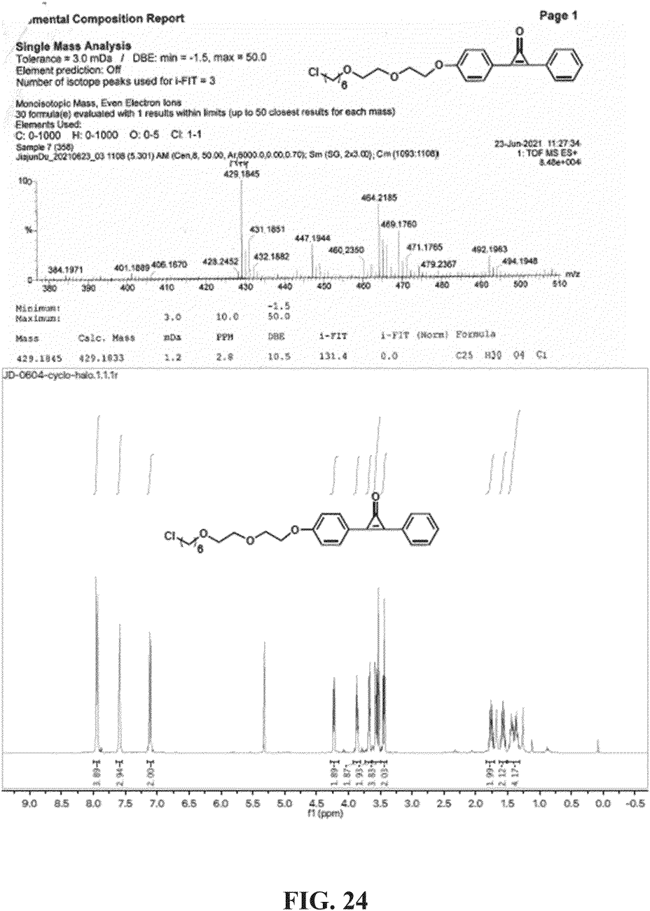

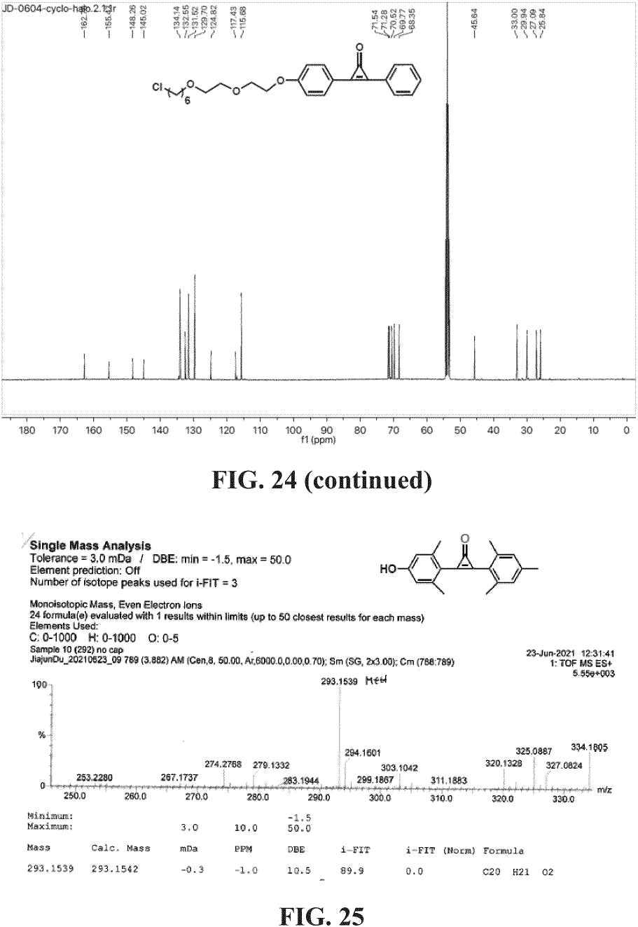

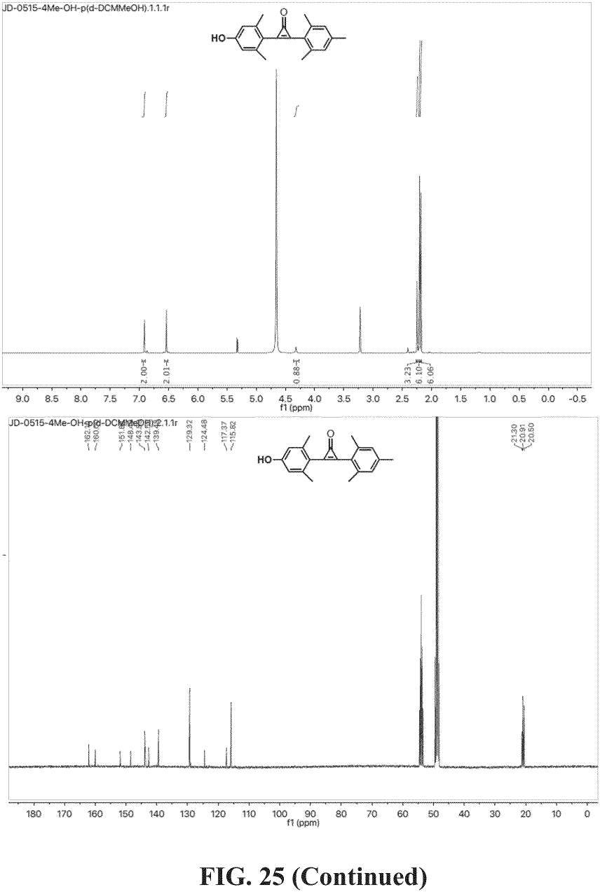

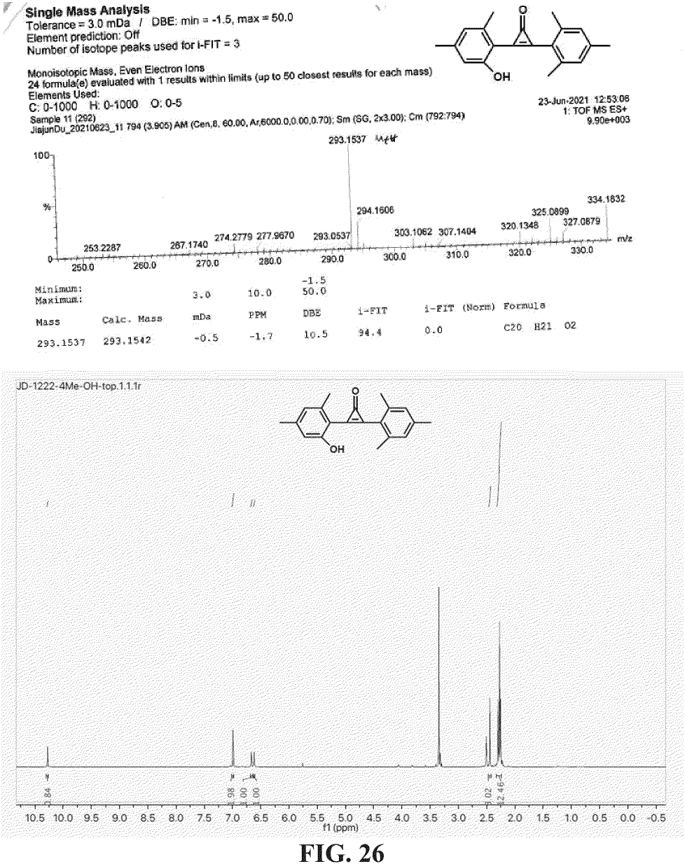

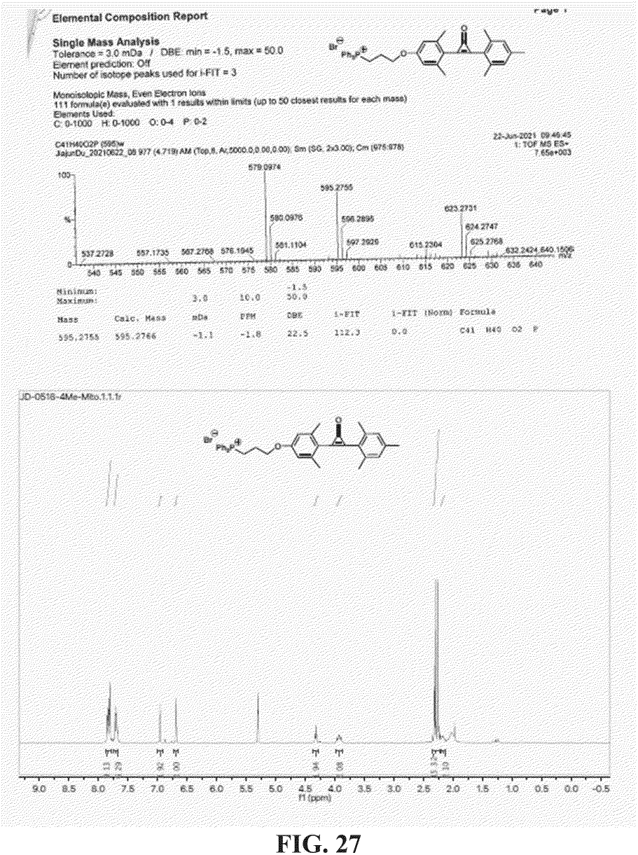

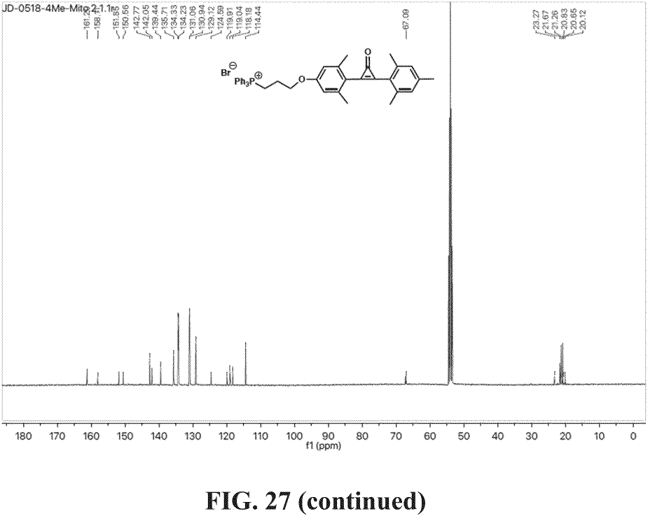

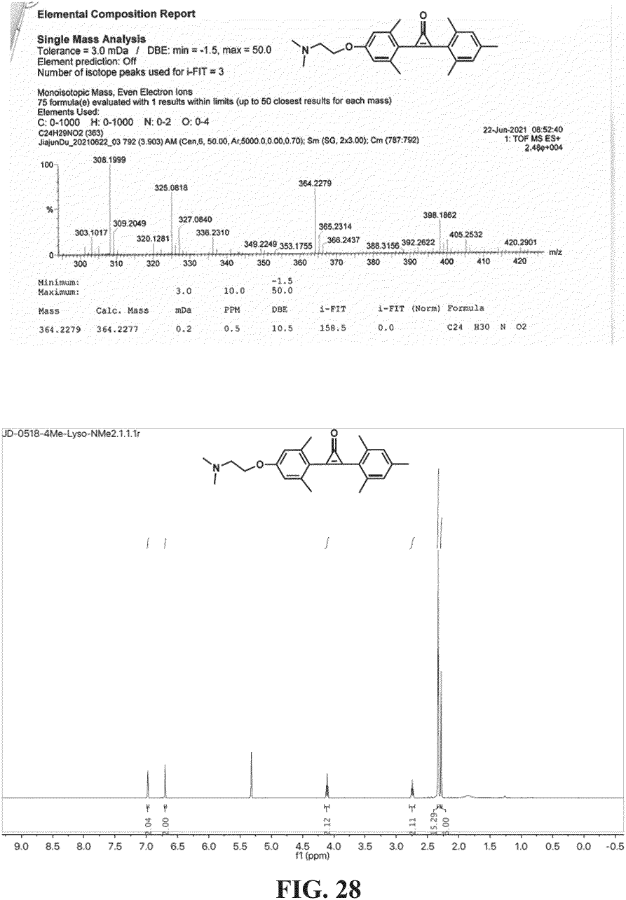

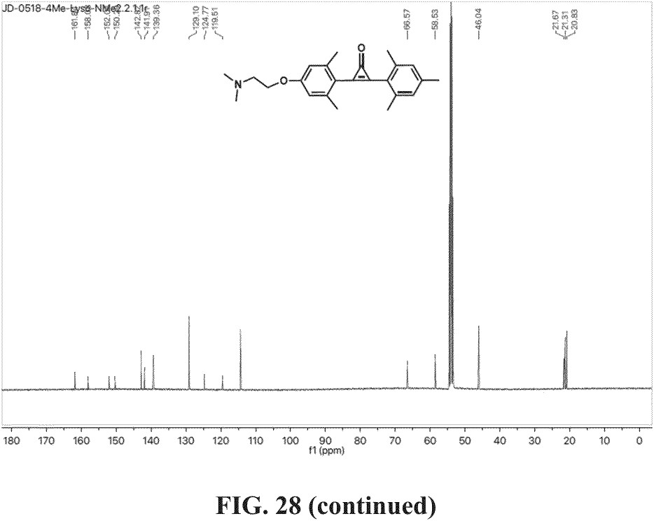

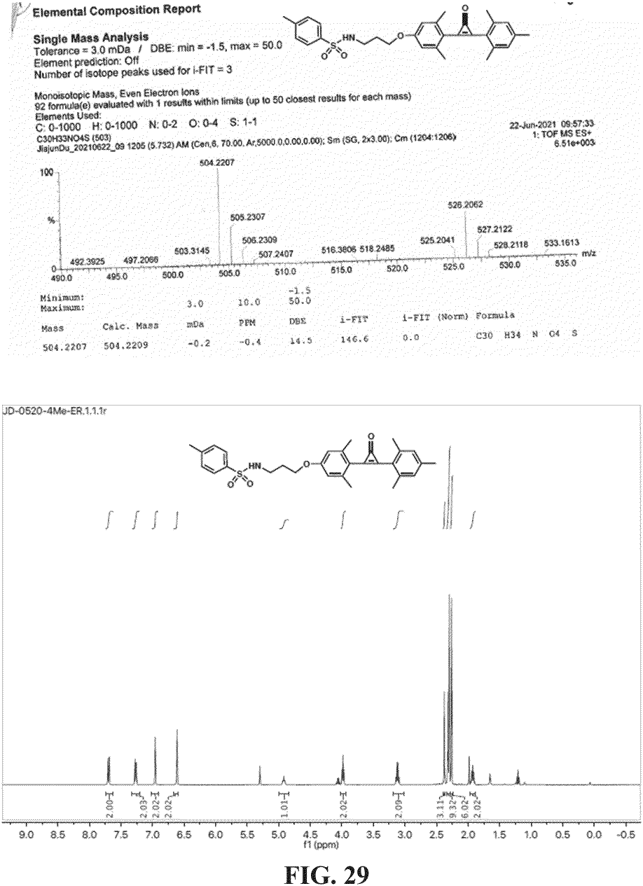

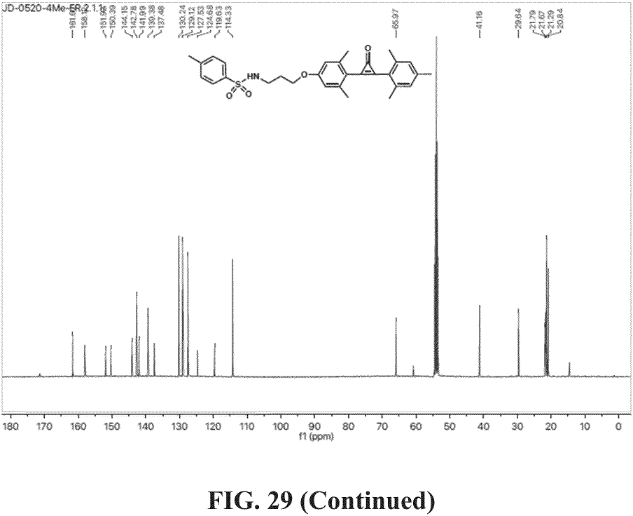

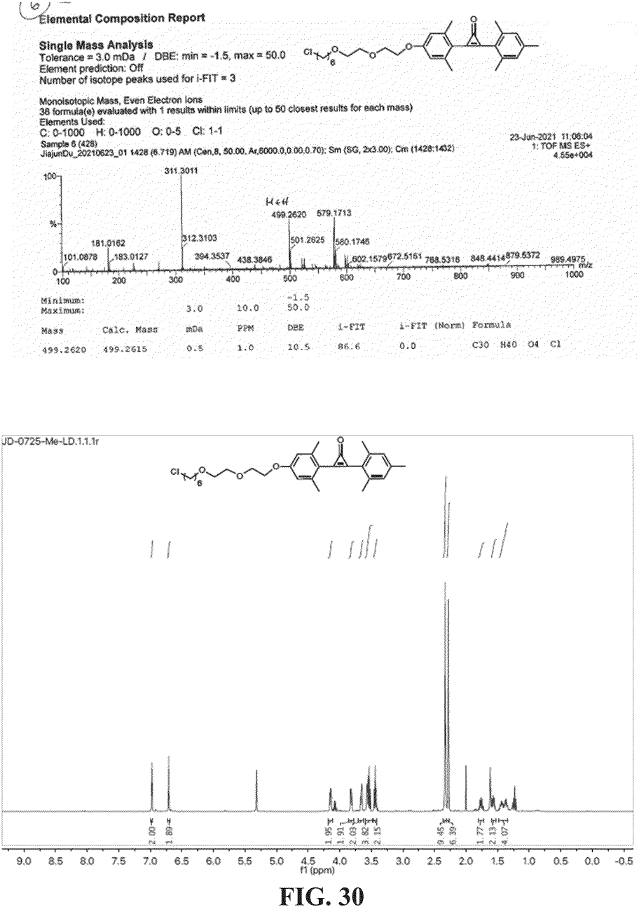

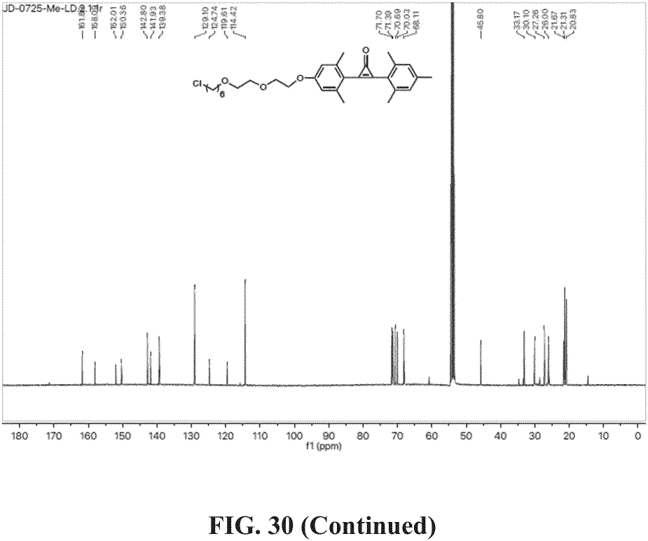

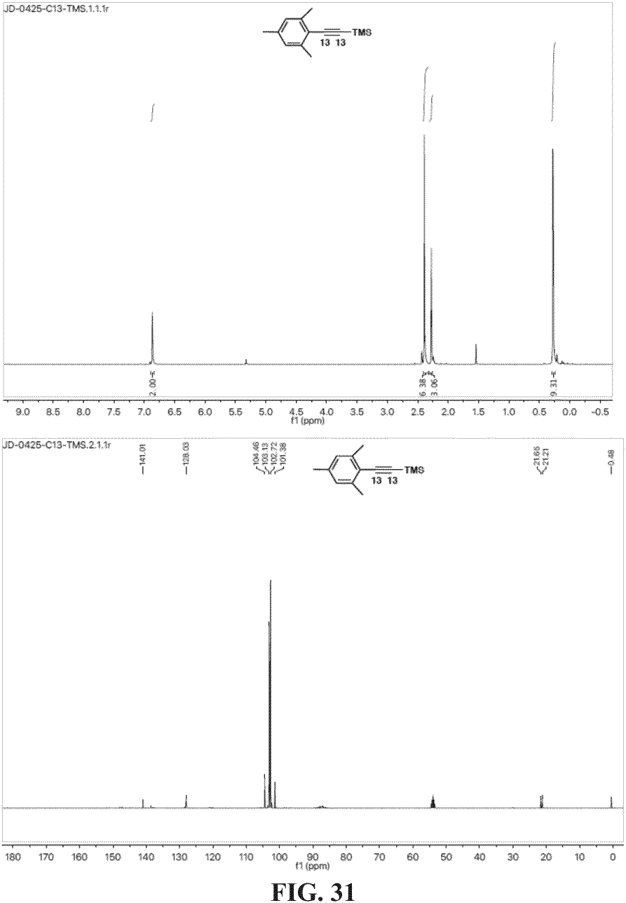

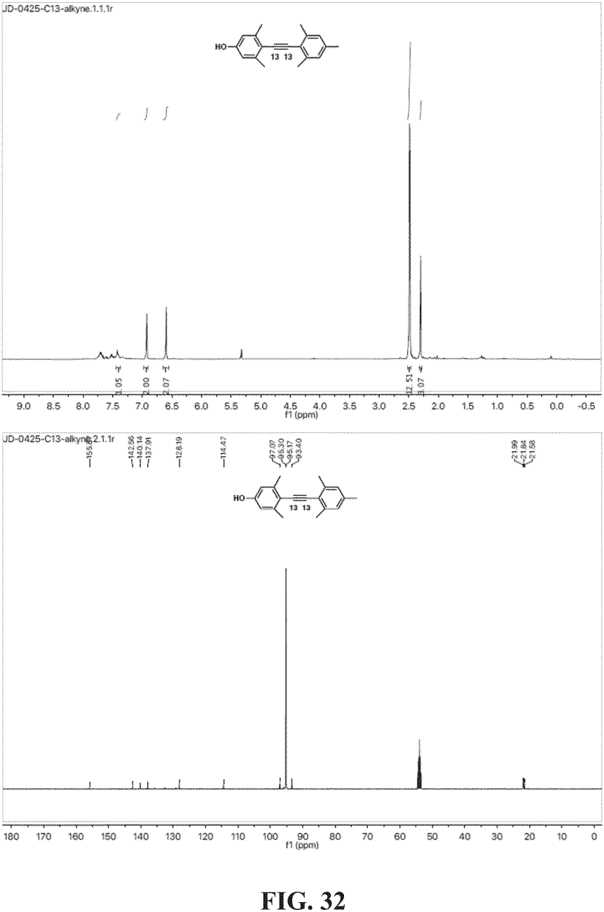

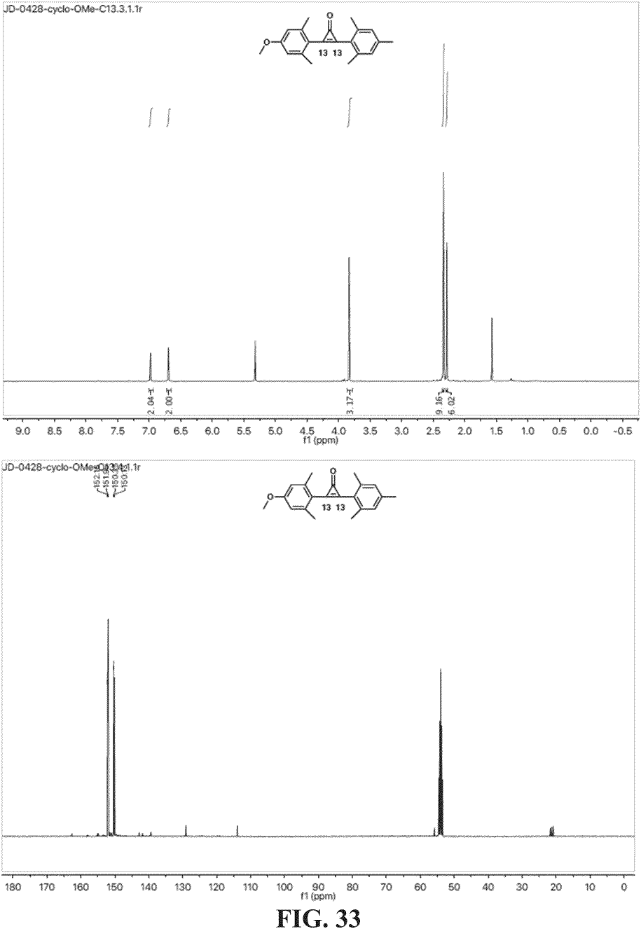

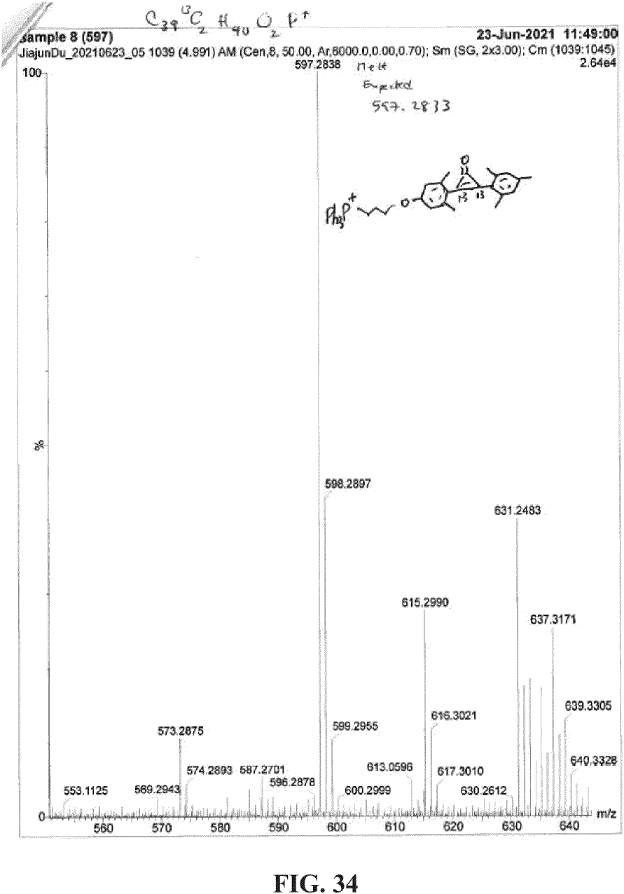

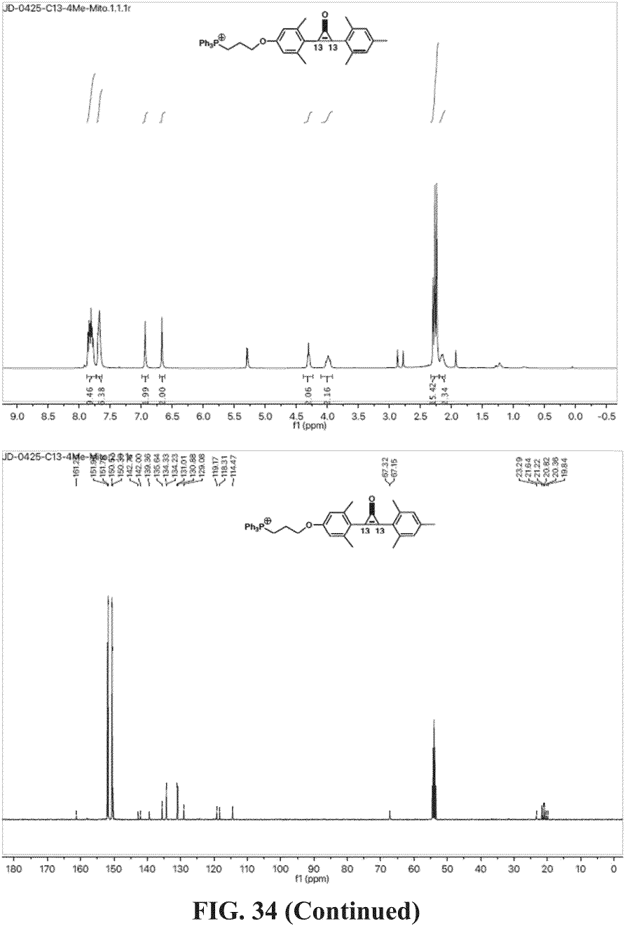

FIG. 21 presents two independent sets of cells labeled with Cyclo-LD and Me-Lyso, which were pulsed with 405 nm activation (selective activation of two cells in FIG. 21, panel a, zoom-in activation in FIG. 21, panel b) and chased with rapamycin inhibition. Live HeLa cells were pretreated with 100 µM oleic acid for 2 hours to induce droplet formation and then labeled with Cyclo-LD and Me-Lyso. The cells were then treated with 5 µM rapamycin, the mTOR inhibitor. The cells were activated by 405 nm (selective activation of two cells indicated by the dashed white boxes in (a), zoom-in activation in (b)) immediately after rapamycin treatment (0 min). Sequential timelapse SRS imaging was performed in both lipid droplets (LD) and lysosomes (Lyso) channels at 0 min (left) and 30 min (right) after photoactivation. Scale bar: 10 µm.

At the beginning of rapamycin incubation (0 min), there are minimum colocalization between lipid droplets and lysosomes (LD/Lyso₀ ₘᵢₙ). After 30 minutes, high colocalization between these two organelles from the activated cells (LD/Lyso₃₀ ₘᵢₙ) was observed. Interestingly, the co-localization was mainly driven by the motion of lysosomes to the lipid droplets, which further underscores the value of multicolor Raman organelle probes for deciphering dynamic processes.

Going above photoactivatable Raman imaging contrast, cyclopropenones also show intense infrared (IR) absorption peaks around 1900 cm⁻¹. This further provides contrast for IR spectroscopy and microscopy, and is highly beneficial for IR-Raman dual mode analysis.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A photoactivatable vibrational probe, having a structure according to Formula I: 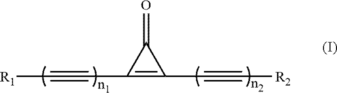
wherein R₁ and R₂ independently represent a monocyclic or polycyclic, aromatic or heteroaromatic ring; n₁ and n₂ are independently 0 or 1, and
wherein the photoactivatable vibrational probe upon photoactivation forms a vibrational probe comprising a planar alkyne generated from the cyclopropenone of Formula I.

2. The photoactivatable vibrational probe of claim 1, having a structure according to Formula II: 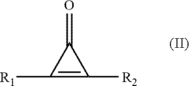
.

3. The photoactivatable vibrational probe of claim 2, wherein R₁ and R₂ each is a phenyl group.

4. The photoactivatable vibrational probe of claim 3, wherein one or more carbon atoms of one or both phenyl groups are substituted with a substituent selected from the group consisting of: alkyl, aryl, heteroaryl, halogen, hydroxyl, alkyl alcohol, carboxyl, alkoxyl, aryloxyl, thio, alkythio, amino, alkylamino, aldehyde, alkenyl, alkynyl, benzyl, carboxamide, azo, ester, carbonyl, nitrile, nitro, phenyl, sulfonyl, sulfinyl, and a combination thereof.

5. The photoactivatable vibrational probe of claim 1, having a formula of: 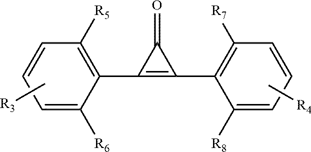
wherein R
₃ and R₄ are independently a hydrogen, a hydroxyl group, an alkyl group, an alkoxy group, an amide, a ketone, a carboxyl group, a halogen, an ether, or an ester; R₅, R₆, R₇ and R₈ are independently hydrogen, alkyl, aryl, heteroaryl, halogen, hydroxyl, alkyl alcohol, carboxyl, alkoxyl, aryloxyl, thio, alkythio, amino, and alkylamino, aldehyde, alkenyl, alkynyl, benzyl, carboxamide, azo, ester, carbonyl, nitrile, nitro, phenyl, sulfonyl, or sulfinyl.

6. The photoactivatable vibrational probe of claim 1, having a formula of: 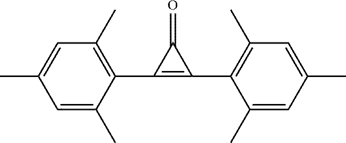
wherein one of the methyl groups in one or both of the phenyl group is unsubstituted or substituted with a hydroxyl group or a hydrogen.

7. (canceled)

8. The photoactivatable vibrational probe of claim 1, further comprising a targeting moiety.

9. The photoactivatable vibrational probe of claim 8, the targeting moiety is covalently attached to the photoactivatable vibrational probe via a hydroxyl group.

10. The photoactivatable vibrational probe of claim 1, having a formula of: 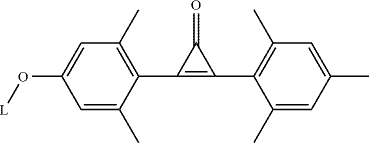
wherein L represents a targeting moiety.

11. The photoactivatable vibrational probe of claim 10, having one of the following structures: 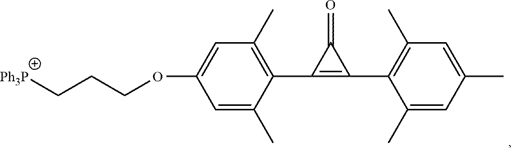 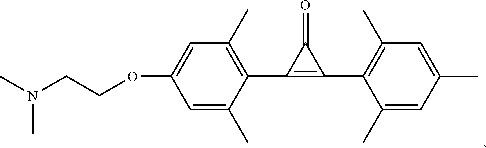 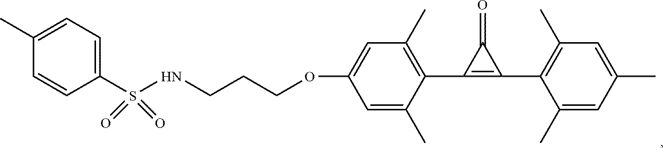 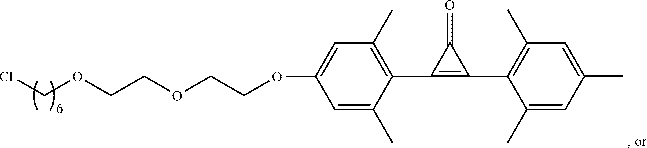 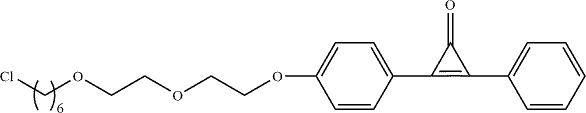
.

12. The photoactivatable vibrational probe of claim 1, comprising at least one ¹³C atom.

13. The photoactivatable vibrational probe of claim 1, having a formula of 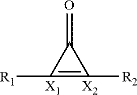
wherein one or both of X
₁ and X₂ is a ¹³C atom.

14. A method of imaging a biological material, comprising:
introducing at least one photoactivatable vibrational probe of claim 1 to the biological material;
activating the at least one photoactivatable vibrational probe with light to generate at least one vibrational probe; and
detecting the at least one vibrational probe using Raman scattering.

15. The method of claim 14, wherein the contacting comprising introducing two or more photoactivatable vibrational probes into the biological material, wherein the two or more photoactivatable vibrational probes when activated exhibit different Raman peaks.

16. The method of claim 14, wherein the at least one photoactivatable vibrational probe is attached to a targeting moiety, and the target moiety is capable of specifically binding to a cell marker, an organelle, proteins, sugars, lipids, nucleic acids or metabolites.

17. The method of claim 16, wherein the targeting moiety binds to a receptor of an organelle or a cell in the biological material.

18. The method of claim 17, wherein the targeting moiety targets mitochondria, lysosome, endoplasmic reticulum or lipid droplet.

19. The method of claim 16, wherein the two or more photoactivatable vibrational probes each is attached to a targeting moiety that targets a different cell type, a different organelle or a different biomolecule in the biological material.

20. The method of claim 14, wherein activating the at least one photoactivable vibrational probe comprises applying light having a wavelength from about 200 nm to about 1500 nm through one-photon or multi-photon absorption to the biological material.

21. The method of claim 14, wherein one or more of the at least one vibrational probe comprises an isotopically modified alkyne .

22. The method of claim 14, wherein the at least one vibrational probe is imaged using a stimulated Raman scattering (SRS) imaging procedure or spontaneous Raman imaging procedure.

23. The method of claim 14, wherein detecting the at least one vibrational probe using Raman scattering comprises
detecting the at least one vibrational probe at a first time point;
detecting the at least one vibrational probe at a second time point, wherein the first time point is different from the second time point; and
comparing a first image obtained from detecting the at least one vibrational probe at the first time point and a second image obtained from detecting the at least one vibrational probe at the second time point.

24. (canceled)

25. The method of claim 14, wherein the biological material comprises live cells, biomolecules, a cell line, cells constituent derived from or located in a mammal, organs, living organism, biological tissues, a biological fluid, or a combination thereof.

26. (canceled)

27. (canceled)

28. The method of claim 14, wherein the biological material comprises healthy, diseased or malignant tissue.

29. The method of claim 14, wherein the contacting occurs in vivo, ex vivo, or in vitro.

30-43. (canceled)
